(19) United States
(12) Patent Application Publication
(10) Pub. No.: US 2007/0238135 A1
(43) Pub. Date: Oct. 11, 2007

(54) NOVEL G-PROTEIN COUPLED RECEPTOR AND DNA SEQUENCES THEREOF

(76) Inventor: Klemens KAUPMANN, Basel (CH)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 11/762,237

(22) Filed: Jun. 13, 2007

Related U.S. Application Data
(63) Continuation of application No. 10/485,006, filed on Jan. 27, 2004, now abandoned.
(60) Provisional application No. 60/315,111, filed on Aug. 27, 2001.

(51) Int. Cl. C07K 14/00; C07H 21/04; C07K 16/28; C12N 1/00; C12N 15/63; G01N 33/53

(52) U.S. Cl. 435/7.2; 435/243; 435/320.1; 436/501; 530/350; 530/387.1; 530/387.3; 536/23.5

(57) ABSTRACT

This invention relates to newly identified polypeptides and polynucleotides encoding such polypeptides, to their use in diagnosis and in identifying compounds that may be agonists, antagonists that are potentially useful in therapy, and to production of such polypeptides and polynucleotides, belonging to the class of G-protein coupled receptors.

### FIELD OF THE INVENTION

This invention relates to newly identified polypeptides and polynucleotides encoding such polypeptides, to their use in diagnosis and in identifying compounds that may be agonists or antagonists that are potentially useful in therapy, and to production of such polypeptides and polynucleotides, sharing similarity to G-protein coupled receptors (GPCR).

### BACKGROUND OF THE INVENTION

The drug discovery process is currently undergoing a fundamental revolution as it embraces “functional genomics”, that is, high throughput genome- or gene-based biology. This approach as a means to identify genes and gene products as therapeutic targets is rapidly superseding earlier approaches based on “positional cloning”. A phenotype, that is a biological function or genetic disease, would be identified and this would then be tracked back to the responsible gene, based on its genetic map position.

Functional genomics relies heavily on high-throughput DNA sequencing technologies and the various tools of bioinformatics to identify gene sequences of potential interest from the many molecular biology databases now available. There is a continuing need to identify and characterise further genes and their related polypeptides/proteins, as targets for drug discovery.

### SUMMARY OF THE INVENTION

The present invention relates to purified metabotropic glutamate receptor related membrane receptor proteins of human origin referred to herein as human mGRR1a, mGRR1b and mGRR2 (all three are herein referred to as mGRR). The similarity of mGRR to metabotropic glutamate receptors suggests that these receptors also belong to family 3 of the GPCR. This receptor family comprises metabotropic glutamate receptors, GABA-B receptors, the Ca-sensing receptor, putative taste receptors and a family of olfactory vomeronasal receptors. The closest mammalian GPCR homologue found to the polypeptides of the invention in public domain databases is the metabotropic glutamate receptor type 3 (accession Q11923) with 23% identical and 43% similar amino acid residues to the polypeptide of mGRR1a described in SEQ ID NO: 2. Such polypeptides and polynucleotides are of interest in relation to methods of treatment of certain diseases, including, but not limited to the treatment of disorders associated with the central and peripheral nervous systems. In particular, mGRR receptor agonists or antagonists can e.g. be useful in treating neurological and/or psychiatric diseases, including but not limited to dementia, schizophrenia, depression, affective disorders, epilepsy, and motoric disorders, hereinafter referred to as “diseases of the invention”. In a further aspect, the invention relates to methods for identifying agonists and antagonists to mGRR (e.g., inhibitors) using the materials provided by the invention, and treating conditions associated with imbalance of such identified compounds. In still a further aspect, the invention relates to diagnostic assays for detecting diseases associated with inappropriate mGRR activity or levels.

### DESCRIPTION OF THE INVENTION

Glossary

The following definitions are provided to facilitate understanding of certain terms used herein.

“Isolated” means altered from its natural state, i.e. if it occurs in nature, it has been changed or removed from its original environment, or both. For example, a polynucleotide or a polypeptide naturally present in a living organism is not “isolated,” but the same polynucleotide or polypeptide separated from the coexisting materials of its natural state is “isolated”, as the term is employed herein. Moreover, a polynucleotide or polypeptide that is introduced into an organism by transformation, genetic manipulation or by any other recombinant method is “isolated” even if it is still present in said organism, which organism may be living or non-living.

“Polynucleotide” generally refers to any polyribonucleotide (RNA) or polydeoxribonucleotide (DNA), which may be unmodified or modified RNA or DNA. “Polynucleotides” include, without limitation, single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, “polynucleotide” refers to triple-stranded regions comprising RNA or DNA or both RNA and DNA. The term “polynucleotide” also includes DNAs or RNAs containing one or more modified bases and DNAs or RNAs with backbones modified for stability or for other reasons.

“Modified” bases include, for example, tritylated bases and unusual bases such as inosine. A variety of modifications may be made to DNA and RNA; thus, “polynucleotide” embraces chemically, enzymatically or metabolically modified forms of polynucleotides as typically found in nature, as well as the chemical forms of DNA and RNA characteristic of viruses and cells. “Polynucleotide” also embraces relatively short polynucleotides, often referred to as oligonucleotides.

“Polypeptide” refers to any polypeptide comprising two or more amino acids joined to each other by peptide bonds or modified peptide bonds, i.e., peptide isosteres. “Polypeptide” refers to both short chains, commonly referred to as peptides, oligopeptides or oligomers, and to longer chains, generally referred to as proteins. Polypeptides may contain amino acids other than the 20 gene-encoded amino acids. “Polypeptides” include amino acid sequences modified either by natural processes, such as post-translational processing, or by chemical modification techniques that are well known in the art. Such modifications are well described in basic texts and in more detailed monographs, as well as in a voluminous research literature familiar to one of skill in the art. Modifications may occur anywhere in a polypeptide, including the peptide backbone, the amino acid side-chains and the amino or carboxyl termini. It will be appreciated that the same type of modification may be present to the same or varying degrees at several sites in a given polypeptide. Also, a given polypeptide may contain many types of modifications. Polypeptides may be branched as a result of ubiquitination, and they may be cyclic, with or without branching. Cyclic, branched and branched cyclic polypeptides may result from post-translation natural processes or may be made by synthetic methods. Modifications include acetylation, acylation, ADP-ribosylation, amidation, biotinylation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cystine, formation of pyroglutarnate, formylation, gamma-carboxylation, glycosylation, GPl anchor formation, hydroxylation, iodination, methylation, myristoylation, oxidation, proteolytic processing, phosphorylation, prenylation, racernization, selenoylation, sulfation, transfer-RNA mediated addition of amino acids to proteins such as arginylation, and ubiquitination (see, for instance, Proteins—Structure and Molecular Properties, 2nd Ed., T. E. Creighton, W.H. Freeman and Company, New York, 1993; Wold, F., Post-translational Protein Modifications: Perspectives and Prospects, 1-12, in Post-translational Covalent Modification of Proteins, B. C. Johnson, Ed., Academic Press, New York, 1983; Seifter et aL, “Analysis for protein modifications and nonprotein cofactors”, Meth Enzymol, 182, 626-646, 1990, and Rattan et al., “Protein Synthesis: Post-translational Modifications and Aging”, Ann NY Acad Sci, 663, 48-62, 1992).

“Fragment” of a polypeptide sequence refers to a polypeptide sequence that is shorter than the reference sequence but that retains essentially the same biological function or activity as the reference polypeptide. “Fragment” of a polynucleotide sequence refers to a polynucleotide sequence that is shorter than the reference sequence of SEQ ID NO: 1, SEQ ID NO: 3 or SEQ ID NO: 4.

“mGRR” refers to the two splice variants human mGRR1a and mGRR1b and human mGRR2. The N-terminal sequence of mGRR1b (amino acid 1 to 53) does not share significant sequence similarity to the N-terminal sequence of mGRR1a (amino acid 1 to 82). The human mGRR2 amino acid sequence has 59% identity to human mGRR1a (bestfit alignment residues 61-733 of mGRR2 with 81-765 of mGRR1a).

“Variant” refers to a polynucleotide or polypeptide that differs from a reference polynucleotide or polypeptide, but retains the essential properties thereof. A typical variant of a polynucleotide differs in nucleotide sequence from the reference polynucleotide. Changes in the nucleotide sequence of the variant may or may not alter the amino acid sequence of a polypeptide encoded by the reference polynucleotide. Nucleotide changes may result in amino acid substitutions, additions, deletions, fusions and truncations in the polypeptide encoded by the reference sequence, as discussed below. A typical variant of a polypeptide differs in amino acid sequence from the reference polypeptide. Generally, alterations are limited so that the sequences of the reference polypeptide and the variant are closely similar overall and, in many regions, identical. A variant and reference polypeptide may differ in amino acid sequence by one or more substitutions, insertions, deletions in any combination. A substituted or inserted amino acid residue may or may not be one encoded by the genetic code. Typical conservative substitutions include Gly, Ala; Val, lie, Leu; Asp, Glu; Asn, Gln-l Ser. Thr; Lys, Arg; and Phe and Tyr. A variant of a polynucleotide or polypeptide may be naturally occurring such as an allele, or it may be a variant that is not known to occur naturally. Non-naturally occurring variants of polynucleotides and polypeptides may be made by mutagenesis techniques or by direct synthesis. Also included as variants are polypeptides having one or more post-translational modifications, for instance glycosylation, phosphorylation, methylation, ADIP ribosylation and the like. Embodiments include methylation of the N-terminal amino acid, phosphorylations of serines and threonines and modification of C-terminal glycines.

“Polymorphism” refers to a variation in nucleotide sequence (and encoded polypeptide sequence, if relevant) at a given position in the genome within a population.

“Single Nucleotide Polymorphism” (SNP) refers to the occurence of nucleotide variability at a single nucleotide position in the genome, within a population. An SNP may occur within a gene or within intergenic regions of the genome. SNPs can be assayed using Allele Specific Amplification (ASA). For the process at least 3 primers are required. A common primer is used in reverse complement to the polymorphism being assayed. This common primer can be between 50 and 1500 bps from the polymorphic base. The other two (or more) primers are identical to each other except that the final 3′ base wobbles to match one of the two (or more) alleles that make up the polymorphism. Two (or more) PCR reactions are then conducted on sample DNA, each using the common primer and one of the Allele Specific Primers.

“Splice Variant” as used herein refers to cDNA molecules produced from RNA molecules initially transcribed from the same genomic DNA sequence but which have undergone alternative RNA splicing. Alternative RNA splicing occurs when a primary RNA transcript undergoes splicing, generally for the removal of introns, which results in the production of more than one mRNA molecule each of that may encode different amino acid sequences. The term splice variant also refers to the proteins encoded by the above cDNA molecules.

“Identity” reflects a relationship between two or more polypeptide sequences or two or more polynucleotide sequences, determined by comparing the sequences. In general, identity refers to an exact nucleotide to nucleotide or amino acid to amino acid correspondence of the two polynucleotide or two polypeptide sequences, respectively, over the length of the sequences being compared.

“% Identity” —For sequences where there is not an exact correspondence, a “% identity” may be determined. In general, the two sequences to be compared are aligned to give a maximum correlation between the sequences. This may include inserting “gaps” in either one or both sequences, to enhance the degree of alignment. A % identity may be determined over the whole length of each of the sequences being compared (so-called global alignment), that is particularly suitable for sequences of the same or very similar length, or over shorter, defined lengths (so-called local alignment), that is more suitable for sequences of unequal length.

“Similarity” is a further, more sophisticated measure of the relationship between two polypeptide sequences. In general, “similarity” means a comparison between the amino acids of two polypeptide chains, on a residue by residue basis, taking into account not only exact correspondences between a between pairs of residues, one from each of the sequences being compared (as for identity) but also, where there is not an exact correspondence, whether, on an evolutionary basis, one residue is a likely substitute for the other. This likelihood has an associated “score” from which the “% similarity” of the two sequences can then be determined.

Methods for comparing the identity and similarity of two or more sequences are well known in the art. Thus for instance, programs available in the Wisconsin Sequence Analysis Package, version 9.1 (Devereux J et al, Nucleic Acids Res, 12, 387-395, 1984, available from Genetics Computer Group, Madison, Wis., USA), for example the programs BESTFIT and GAP, may be used to determine the % identity between two polynucleotides and the % identity and the % similarity between two polypeptide sequences. BESTFIT uses the “local homology” algorithm of Smith and Waterman (J Mol Biol, 147, 195-197, 1981, Advances in Applied Mathematics, 2, 482-489, 1981) and finds the best single region of similarity between two sequences. BESTFIT is more suited to comparing two polynucleotide or two polypeptide sequences that are dissimilar in length, the program assuming that the shorter sequence represents a portion of the longer. In comparison, GAP aligns two sequences, finding a “maximum similarity”, according to the algorithm of Neddleman and Wunsch (J Mol Biol, 48, 443-453, 1970). GAP is more suited to comparing sequences that are approximately the same length and an alignment is expected over the entire length.

Preferably, the parameters “Gap Weight” and “Length Weight” used in each program are 50 and 3, for polynucleotide sequences and 12 and 4 for polypeptide sequences, respectively. Preferably, % identities and similarities are determined when the two sequences being compared are optimally aligned.

Other programs for determining identity and/or similarity between sequences are also known in the art, for instance the BLAST family of programs (Altschul S F et al, J Mol Biol, 215, 403-410, 1990, Altschul S F et al, Nucleic Acids Res., 25:389-3402, 1997, available from the National Center for Biotechnology Information (NCBI), Bethesda, Md., USA and accessible through the home page of the NCBI at www.ncbi.nlm.nih.gov) and FASTA (Pearson W R. Methods in Enzymology, 183, 63-99, 1990; Pearson W R and Lipman D J. Proc Nat Acad Sci USA, 85, 2444-2448, 1988, available as part of the Wisconsin Sequence Analysis Package).

Preferably, the BLOSUM62 amino acid substitution matrix (Henikoff S and Henikoff J G. Proc. Nat. Acad Sci. USA, 89, 10915-10919, 1992) is used in polypeptide sequence comparisons including where nucleotide sequences are first translated into amino acid sequences before comparison.

Preferably, the program BESTFIT is used to determine the % identity of a query polynucleotide or a polypeptide sequence with respect to a reference polynucleotide or a polypeptide sequence, the query and the reference sequence being optimally aligned and the parameters of the program set at the default value, as hereinbefore described.

“Identity Index” is a measure of sequence relatedness which may be used to compare a candidate sequence (polynucleotide or polypeptide) and a reference sequence. Thus, for instance, a candidate polynucleotide sequence having, for example, an Identity Index of 0.95 compared to a reference polynucleotide sequence is identical to the reference sequence except that the candidate polynucleotide sequence may include on average up to five differences per each 100 nucleotides of the reference sequence. Such differences are selected from the group consisting of at least one nucleotide deletion, substitution, including transition and transversion, or insertion. These differences may occur at the 5′ or 3′ terminal positions of the reference polynucleotide sequence or anywhere between these terminal positions, interspersed either individually among the nucleotides in the reference sequence or in one or more contiguous groups within the reference sequence. In other words, to obtain a polynucleotide sequence having an Identity Index of 0.95 compared to a reference polynucleotide sequence, an average of up to 5-25 in every 100 of the nucleotides of the in the reference sequence may be deleted, substituted or inserted, or any combination thereof, as hereinbefore described. The same applies mutatis mutandis for other values of the Identity Index, for instance 0.96, 0.97, 0.98 and 0.99.

Similarly, for a polypeptide, a candidate polypeptide sequence having, for example, an Identity Index of 0.95 compared to a reference polypeptide sequence is identical to the reference sequence except that the polypeptide sequence may include an average of up to five differences per each 100 amino acids of the reference sequence. Such differences are selected from the group consisting of at least one amino acid deletion, substitution, including conservative and non-conservative substitution, or insertion. These differences may occur at the amino- or carboxy-terminal positions of the reference polypeptide sequence or anywhere between these terminal positions, interspersed either individually among the amino acids in the reference sequence or in one or more contiguous groups within the reference sequence. In other words, to obtain a polypeptide sequence having an Identity Index of 0.95 compared to a reference polypeptide sequence, an average of up to 5 in every 100 of the amino acids in the reference sequence may be deleted, substituted or inserted, or any combination thereof, as hereinbefore described. The same applies mutatis mutandis for other values of the Identity Index, for instance 0.96, 0.97, 0.98 and 0.99.

The relationship between the number of nucleotide or amino acid differences and the Identity Index may be expressed in the following equation:
nₐ≦xₐ−(xₐ·I)
in which:

nₐ is the number of nucleotide or amino acid differences,

xₐ is the total number of nucleotides or amino acids in SEQ ID NO: 1 or SEQ ID NO: 2 for mGRR1a, respectively SEQ ID NO: 4 or SEQ ID NO: 5 for mGRR1b

I is the Identity Index,

·is the symbol for the multiplication operator, and in which any non-integer product of xₐ and I is rounded down to the nearest integer prior to subtracting it from xₐ.

“Homolog” is a generic term used in the art to indicate a polynucleotide or polypeptide sequence possessing a high degree of sequence relatedness to a reference sequence. Such relatedness may be quantified by determining the degree of identity and/or similarity between the two sequences as hereinbefore defined. Falling within this generic term are the terms “ortholog”, and “paralog”. “Ortholog” refers to a polynucleotide or polypeptide that is the functional equivalent of the polynucleotide or polypeptide in another species. “Paralog” refers to a polynucleotideor polypeptide that within the same species which is functionally similar.

“Fusion protein” refers to a protein encoded by two, unrelated, fused genes or fragments thereof. Examples have been disclosed in U.S. Pat. Nos. 5,541,087, 5,726,044. In the case of Fc-mGRR, employing an immunoglobulin Fc region as a part of a fusion protein is advantageous for performing the functional expression of Fc-mGRR or fragments of mGRR, to improve pharmacokinetic properties of such a fusion protein when used for therapy and to generate a dimeric Fc-mGRR. The Fc-mGRR DNA construct may comprise in 5′ to 3′ direction, a secretion cassette, i.e. a signal sequence that triggers export from a mammalian cell, DNA encoding an immunoglobulin Fc region fragment, as a fusion partner, and a DNA encoding Fc-mGRR or fragments thereof. In some uses it would be desirable to be able to alter the intrinsic functional properties (complement binding, Fc-Receptor binding) by mutating the functional Fc sides while leaving the rest of the fusion protein untouched or delete the Fc part completely after expression.

In a first aspect, the present invention provides mGRR polypeptides.

Such polypeptides comprise:
(a) an isolated mGRR polypeptide encoded by a polynucleotide comprising the sequence of SEQ ID NO: 1, SEQ ID NO: 3 or SEQ ID NO: 4;
(b) an isolated mGRR polypeptide comprising a polypeptide sequence having at least 80%, 90%, 95%, 98%, or 99% identity to the polypeptide sequence of SEQ ID NO: 2 or SEQ ID NO: 5 and which shows properties in the ligand binding assay similar to those of mGRR;
(c) an isolated mGRR1a or mGRR1b polypeptide comprising the polypeptide sequence of SEQ ID NO: 2 or SEQ ID NO: 5;
(d) an isolated mGRR polypeptide having at least 80%, 90%, 95%, 98%, or 99% identity to the polypeptide sequence of SEQ ID NO: 2 or SEQ ID NO: 5 and which shows properties in the ligand binding assay similar to those of mGRR;
(e) the polypeptide sequence of SEQ ID NO: 2 or SEQ ID NO: 5;
(f) an isolated mGRR polypeptide having or comprising a polypeptide sequence that has an Identity Index of 0.80, 0.90, 0.95, 0.98, or 0.99 compared to the polypeptide sequence of SEQ ID NO: 2 or SEQ ID NO: 5 and which shows properties in the ligand binding assay similar to those of mGRR; or
(g) a fragment or variant of such a polypeptide according to (a) to (f).

Similar properties in the ligand binding assay means that under the same conditions for buffers, ions, pH and other modulators such as nucleotides, the detectable signal to noise ratio is in the range of +/−30% of the signal of the mGRR polypeptide.

Polypeptides of the present invention are members of the G protein-coupled receptors family of polypeptides. The observed brain specific regional distribution of mGRR1mRNA (Table 1) provides information on indications for mGRR ligands. mGRR ligands include the natural ligand as well as modulators of mGRR activity, such as anti-mGRR antibodies and/or small molecules that agonize or antagonize mGRR-mediated signalling. The brain specific regional distribution of mGRR mRNA indicates the importance of mGRRs in the regulation of normal brain function. Thus, abnormalities in the expression, abundance or activity of these polypeptides could lead to a wide variety of neurological and and/or psychiatric diseases, including, but not limited to, dementia, schizophrenia, depression, affective disorders, epilepsy, and motoric disorders. For example, in Alzheimer's disease and other dementias such as Age Associated Memory Impairment and Multi Infarct Dementia, loss of cognitive function is associated with reduced levels of a number of neurotransmitters in the brain.

Family 3 GPCR such as metabotropic glutamate receptors and GABA-B receptors also modulate synaptic transmission and in comparison to ion channel modulators their effects are rather long-lasting and modulatory. All these indications are hereinafter referred to as “biological activity” of mGRR1a,mGRR1b or mGRR2. Preferably, a polypeptide of the present invention exhibits at least one biological activity characterisitc of mGRR1a, mGRR1b or mGRR2.

Polypeptides of the present invention also include variants of the aforementioned polypeptides, including all allelic forms and splice variants. Such polypeptides vary from the reference polypeptide by insertions, deletions, and substitutions that may be conservative or non-conservative, or any combination thereof.

Preferred fragments of polypeptides of the present invention include an isolated polypeptide comprising an amino acid sequence having at least 30, 50 or 100 contiguous amino acids from the amino acid sequence of SEQ ID NO: 2, SEQ ID NO: 4 or SEQ ID NO: 6, or an isolated polypeptide comprising an amino acid sequence having at least 30, 50 or 100 contiguous amino acids truncated or deleted from the amino acid sequence of SEQ ID NO: 2, SEQ ID NO: 4 or SEQ ID NO: 6. Preferred fragments are biologically active fragments that mediate the biological activity of mGRR, including those with a similar activity or an improved activity, or with a decreased undesirable activity. Also preferred are those fragments that are antigenic or immunogenic in an animal, especially in a human.

Fragments or variants of the human polypeptides of, SEQ ID NO: 4 or SEQ ID NO: 6 of the invention may be employed for producing the corresponding full-length polypeptide by using the rat or human DNA sequence (either SEQ ID NO: 1, SEQ ID NO: 3, SEQ ID NO: 5 or SEQ ID NO: 16) in low stringency hybridization to isolate the full length rat cDNA. This cDNA allows the generation of mGRR polypeptides using mammalian expression systems (see example 2). Stringency of hybridisation refers to conditions under which polynucleic acids hybrids are stable. Such conditions are evident to those of ordinary skill in the field. As known to those skilled in the art, the stability of hybrids is reflected in the melting temperature (Tₘ) of the hybrid which decreases approximately by 1 to 1.5° C. with every 1% decrease in sequence homology. In general, the stability of a hybrid is a function of sodium ion concentration and temperature. Typically, the hybridisation reaction is performed under conditions of higher stringency, followed by washes of varying stringency. As used herein, high stringency refers to conditions that permit hybridisation of only those nucleic acid sequences that form stable hybrids in 1 M Na⁺ at 65-68° C. High stringency conditions can be provided, for example, by hybridisation in an aqueous solution containing 6×SSC, 5× Denhardt's, 1% SDS (sodium dodecyl sulphate), 0.1 sodium pyrophosphate and 0.1 mg/ml denatured salmon sperm DNA as non specific competitor. Following hybridisation, high stringency washing may be done in several steps, with a final wash (about 30 min) at the hybridisation temperature in 0.2-0.1×SSC, 0.1% SDS. Moderate stringency refers to conditions equivalent to hybridisation in the above described solution but at about 60-62° C. In that case the final wash is performed at the hybridisation temperature in 1×SSC, 0.1% SDS. Low stringency refers to conditions equivalent to hybridisation in the above described solution at about 50-52° C. In that case, the final wash is performed at the hybridisation temperature in 2×SSC, 0.1% SDS. It is understood that these conditions may be adapted and duplicated using a variety of buffers, e.g. formamide-based buffers, and temperatures. Denhardt's solution and SSC are well known to those of skill in the art as are other suitable hybridisation buffers (see, e.g. Sambrook, et al., eds. (1989) Molecular Cloning: A Laboratory Manual, Cold Spring Harbor Laboratory Press, New York or Ausubel, et al., eds. (1990) Current Protocols in Molecular Biology, John Wiley & Sons, Inc.). In particular, the skilled person will understand that the stringency of hybridisation conditions may be varied by altering a number of parameters, primarily the salt concentration and the temperature, and that the conditions obtained are a result of the combined effect of all such parameters. Optimal hybridisation conditions have to be determined empirically, as the length and the GC content of the probe also play a role.

The polypeptides of the present invention may be in the form of the “mature” protein or may be a part of a larger protein such as a precursor or a fusion protein. It is often advantageous to include an additional amino acid sequence that contains secretory or leader sequences, pro-sequences, sequences that aid in purification, for instance multiple histidine residues, or an additional sequence for stability during recombinant production.

Polypeptides of the present invention can be prepared in any suitable manner, for instance by isolation form naturally occurring sources, from genetically engineered host cells comprising expression systems (vide infra) or by chemical synthesis, using for instance automated peptide synthesizers, or a combination of such methods. The means for preparing such polypeptides are well understood in the art.

In a further aspect, the present invention relates to mGRR polynucleotides. Such polynucleotides include:
(a) an isolated mGRR polynucleotide comprising a polynucleotide sequence having at least 80%, 90%, 95%, 98%, or 99% identity to the polynucleotide sequence of SEQ ID NO: 1, SEQ ID NO: 3 or SEQ ID NO: 5 and which encodes for polypeptides which show properties in the ligand binding assay similar to mGRR;
(b) an isolated polynucleotide comprising the polynucleotide of SEQ ID NO: 1, SEQ ID NO: 3 or SEQ ID NO: 5;
(c) an isolated polynucleotide having at least 80%, 90%, 95%, 98%, or 99% identity to the polynucleotide of SEQ ID NO: 1, SEQ ID NO: 3 or SEQ ID NO: 5 and which encodes for polypeptides which show properties in the ligand binding assay similar to mGRR;
(d) the isolated polynucleotide of SEQ ID NO: 1, SEQ ID NO: 3 or SEQ ID NO: 5;
(e) an isolated polynucleotide comprising a polynucleotide sequence encoding a polypeptide sequence having at least 80%, 90%, 95%, 98%, or 99% identity to the polypeptide sequence of SEQ ID NO: 1, SEQ ID NO: 3 or SEQ ID NO: 5 and which encodes for polypeptides which show properties in the ligand binding assay similar to mGRR;
(f) an isolated polynucleotide comprising a polynucleotide sequence encoding the polypeptide of SEQ ID NO: 1, SEQ ID NO: 3 or SEQ ID NO: 5;
(g) an isolated polynucleotide comprising a polynucleotide sequence encoding a polypeptide sequence having at least 80%, 90%, 95%, 98%, or 99% identity to the polypeptide sequence of SEQ ID NO: 1, SEQ ID NO: 3 or SEQ ID NO: 5 and which encodes for polypeptides which show properties in the ligand binding assay similar to mGRR;
(h) an isolated polynucleotide encoding the polypeptide of SEQ ID NO: 1, SEQ ID NO: 3 or SEQ ID NO: 5;
(i) an isolated polynucleotide comprising a polynucleotide sequence that has an Identity Index of 0.80, 0.90, 0.95, 0.98, or 0.99 compared to the polynucleotide sequence of SEQ ID NO: 1, SEQ ID NO: 3 or SEQ ID NO: 5 and which encodes for polypeptides which show properties in the ligand binding assay similar to mGRR; or
k) an isolated polynucleotide comprising a polynucleotide sequence encoding a polypeptide sequence that has an Identity Index of 0.80, 0.90, 0.95, 0.98, or 0.99 compared to the polypeptide sequence of SEQ ID NO: 1 SEQ ID NO: 3 or SED ID NO: 5 and which encode for polypeptides which show properties in the ligand binding assay similar to mGRR; or
I) polynucleotides that are fragments or variants of the above mentioned polynucleotides or that are complementary to above mentioned polynucleotides, over the entire length thereof.

Preferred fragments of polynucleotides of the present invention include an isolated polynucleotide comprising an nucleotide sequence having at least 15, 30, 50 or 100 contiguous nucleotides from the sequence of SEQ ID NO: 1, SEQ ID NO: 3 or SEQ ID NO: 5, or an isolated polynucleotide comprising an sequence having at least 30, 50 or 100 contiguous nucleotides truncated or deleted from the sequence of SEQ ID NO: 1, SEQ ID NO: 3 or SEQ ID NO: 5.

Preferred variants of polynucleotides of the present invention include splice variants, allelic variants, and polymorphisms, including polynucleotides having one or more single nucleotide polymorphisms (SNPs).

Polynucleotides of the present invention also include polynucleotides encoding polypeptide variants that comprise the amino acid sequence of SEQ ID NO: 2, SEQ ID NO: 4 or SEQ ID NO: 6.

In a further aspect, the present invention provides polynucleotides that are RNA transcripts of the DNA sequences of the present invention. Accordingly, there is provided an RNA polynucleotide that:
(a) comprises an RNA transcript of the DNA sequence encoding the polypeptide of SEQ ID NO: 2, SEQ ID NO: 4 or SEQ ID NO: 6;
(b) is the RNA transcript of the, DNA sequence encoding the polypeptide of SEQ ID NO: 2, SEQ ID NO: 4 or SEQ ID NO: 6;
(c) comprises an RNA transcript of the DNA sequence of SEQ ID NO: 1, SEQ ID NO: 3 or SEQ ID NO: 5; or
(d) is the RNA transcript of the DNA sequence of SEQ ID NO: 1, SEQ ID NO: 3 or SEQ ID NO: 5; or
(e) RNA polynucleotides that are complementary thereto.

The polypeptide of the SEQ ID NO: 2 or SEQ ID NO: 4 or SEQ ID NO:6 is related to other proteins of the G protein-coupled receptors family, having homology and/or structural similarity with GPCR—LYMST Jensen, C. P. et al., Proc. Natl. Acad. Sci. U.S.A. 91: 4816-4820, 1994).

Preferred polypeptides and polynucleotides of the present invention are expected to have, inter alia, similar biological functions/properties to their homologous polypeptides and polynucleotides. Furthermore, preferred polypeptides and polynucleotides of the present invention have at least one activity of mGRR.

Polynucleotides of the present invention may be obtained using standard cloning and screening techniques from a cDNA library derived from mRNA in cells of the mammalian brain (see for instant, Sambrook et al., Molecular Cloning: A Laboratory Manual, 2nd Ed., Cold Spring Harbor Laboratory Press, Cold Spring Harbor, N.Y. (1989)). Polynucleotides of the invention can also be obtained from natural sources such as genomic DNA libraries or can be synthesized using well known and commercially available techniques.

When polynucleotides of the present invention are used for the recombinant production of polypeptides of the present invention, the polynucleotide may include the coding sequence for the mature polypeptide, by itself, or the coding sequence for the mature polypeptide in reading frame with other coding sequences, such as those encoding a leader or secretory sequence, a pre-, or pro- or prepro-protein sequence, or other fusion peptide portions. For example, a marker sequence that facilitates purification of the fused polypeptide can be encoded. In certain preferred embodiments of this aspect of the invention, the marker sequence is a hexa-histidine peptide, as provided in the pQE vector (Qiagen, Qiagen AG; Basel, Switzerland) and described in Gentz et aL, Proc Natl Acad Sci USA (1989) 86:821-824, or is an HA tag. The polynucleotide may also contain non-coding 5′ and 3′ sequences, such as transcribed, non-translated sequences, splicing and polyadenylation signals, ribosome binding sites and sequences that stabilize mRNA.

Polynucleotides that are identical, or have sufficient identity to a polynucleotide sequence of SEQ ID NO: 1, SEQ ID NO: 3 or SEQ ID NO: 5 may be used as hybridization probes for cDNA and genomic DNA or as primers for a nucleic acid amplification reaction (for instance, PCR). Such probes and primers may be used to isolate full-length cDNAs and genomic clones encoding polypeptides of the present invention and to isolate cDNA and genomic clones of other genes (including genes encoding paralogs from human sources and orthologs and paralogs from species other than human) that have a high sequence similarity to SEQ ID NO: 1, SEQ ID NO: 3 or SEQ ID NO: 5, typically of at least 95% identity. Preferred probes and primers will generally comprise at least 15 nucleotides, preferably, at least 30 nucleotides and may have at least 50, if not at least 100 nucleotides. Particularly preferred probes will have between 30 and 50 nucleotides. Particularly preferred primers will have between 20 and 25 nucleotides.

A polynucleotide encoding a polypeptide of the present invention, including homologs from species other than human, may be obtained by a process comprising the steps of screening a library under stringent hybridization conditions with a labeled probe having the sequence of SEQ ID NO: 1, SEQ ID NO: 3 or SEQ ID NO: 5 or a fragment thereof, preferably of at least 15 nucleotides; and isolating full-length cDNA and genomic clones containing said polynucleotide sequence. Such hybridization techniques are well known to the skilled artisan. Preferred stringent hybridization conditions include overnight incubation at 42° C. in a solution comprising: 50% formamide, 5×SSC (150 mM NaC, 15 mM trisodium citrate), 50 mM sodium phosphate (pH7.6), 5× Denhardt's solution, 10% dextran sulfate, and 20 microgram/ml denatured, sheared salmon sperm DNA; followed by washing the filters in 0.1×SSC at about 65° C. Thus the present invention also includes isolated polynucleotides, preferably with a nucleotide sequence of at least 100, obtained by screening a library under stringent hybridization conditions with a labeled probe having the sequence of SEQ ID NO: 1,SEQ ID NO: 3 or SEQ ID NO: 5 or a fragment thereof, preferably of at least 15 nucleotides. Human and (partial) rat sequences are 89% identical on the nucleotide level and 89% identical and 90% similar on the amino acid level.

The person skilled in the art will appreciate that, in many cases, an isolated cDNA sequence will be incomplete, in that the region coding for the polypeptide does not extend all the way through to the 5′ terminus. This is a consequence of reverse transcriptase, an enzyme with inherently low “processivity” (a measure of the ability of the enzyme to remain attached to the template during the polymerisation reaction), failing to complete a DNA copy of the mRNA template during first strand cDNA synthesis.

There are several methods available and well known to those skilled in the art to obtain full-length cDNAs, or extend short cDNAs, for example those based on the method of Rapid Amplification of cDNA ends (RACE) (see, for example, Frohman et al., Proc Nat Acad Sci USA 85, 8998-9002, 1988). Recent modifications of the technique, exemplified by the MARATHON technology (Clontech Laboratories Inc, BD Clontech, Basel, Switzerland) for example, have significantly simplified the search for longer cDNAs. In the MARATHON technology, cDNAs have been prepared from mRNA extracted from a chosen tissue and an ‘adaptor’ sequence ligated onto each end. Nucleic acid amplification (PCR) is then carried out to amplify the “missing” 5′ end of the cDNA using a combination of gene specific and adaptor specific oligonucleotide primers. The PCR reaction is then repeated using ‘nested’ primers, that is, primers designed to anneal within the amplified product (typically an adaptor specific primer that anneals further 3′ in the adaptor sequence and a gene specific primer that anneals further 5′ in the known gene sequence). The products of this reaction can then be analysed by DNA sequencing and a full-length cDNA constructed either by joining the product directly to the existing cDNA to give a complete sequence, or carrying out a separate full-length PCR using the new sequence information for the design of the 5′ primer. Recombinant polypeptides of the present invention may be prepared by processes well known in the art from genetically engineered host cells comprising expression systems. Accordingly, in a further aspect, the present invention relates to expression systems comprising a polynucleotide or polynucleotides of the present invention, to host cells which are genetically engineered with such expression sytems and to the production of polypeptides of the invention by recombinant techniques. Cell-free translation systems can also be employed to produce such proteins using RNAs derived from the DNA constructs of the present invention.

For recombinant production, host cells can be genetically engineered to incorporate expression systems or portions thereof for polynucleotides of the present invention. Polynucleotides may be introduced into host cells by methods described in many standard laboratory manuals, such as Davis et al., Basic Methods in Molecular Biology (1986) and Sambrook et al. (ibid).

Preferred methods of introducing polynucleotides into host cells include, for instance, calcium phosphate transfection, DEAE-dextran mediated transfection, transfection, microinjection, cationic lipid-mediated transfection, electroporation, transduction, scrape loading, ballistic introduction or infection.

Representative examples of appropriate hosts include bacterial cells, such as Streptococci, Staphylococci, E coli, Streptomyces and Bacillus subtilis cells; fungal cells, such as yeast cells and Aspergillus cells; insect cells such as Drosophila S2 and Spodoptera Sf9 cells; animal cells such as CHO, COS, HeLa, C127, 3T3, BHK, HEK 293 and Bowes melanoma cells; and plant cells.

A great variety of expression systems can be used, for instance, chromosomal, episomal and virus-derived systems, e.g., vectors derived from bacterial plasmids, from bacteriophage, from transposons, from yeast episomes, from insertion elements, from yeast chromosomal elements, from viruses such as baculoviruses, papova viruses, such as SV40, vaccinia viruses, adenoviruses, fowl pox viruses, pseudorabies viruses and retroviruses, and vectors derived from combinations thereof, such as those derived from plasmid and bacteriophage genetic elements, such as cosmids and phagemids. The expression systems may contain control regions that regulate as well as engender expression. Generally, any system or vector that is able to maintain, propagate or express a polynucleotide to produce a polypeptide in a host may be used. The appropriate polynucleotide sequence may be inserted into an expression system by any of a variety of well-known and routine techniques, such as, for example, those set forth in Sambrook et al (see above). Appropriate secretion signals may be incorporated into the desired polypeptide to allow secretion of the translated protein into the lumen of the endoplasmic reticulum, the periplasmic space or the extracellular environment. These signals may be endogenous to the polypeptide or they may be heterologous signals.

If a polypeptide of the present invention is to be expressed for use in screening assays, it is generally preferred that the polypeptide be produced at the surface of the cell. In this event, the cells may be harvested prior to use in the screening assay. If the polypeptide is secreted into the medium, the medium can be recovered in order to recover and purify the polypeptide. If produced intracellularly, the cells must first be lysed before the polypeptide is recovered.

Polypeptides of the present invention can be recovered and purified from recombinant cell cultures by well-known methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Most preferably, high performance liquid chromatography is employed for purification. Well known techniques for refolding proteins may be employed to regenerate active conformation when the polypeptide is denatured during intracellular synthesis, isolation and/or purification.

Polynucleotides of the present invention may be used as diagnostic reagents, through detecting mutations in the associated gene. Detection of a mutated form of the gene characterised by the polynucleotide of SEQ ID NO: 1, SEQ ID NO: 3 or SEQ ID NO: 5 in the cDNA or genomic sequence and which is associated with a dysfunction will provide a diagnostic tool that can add to, or define, a diagnosis of a disease, or susceptibility to a disease, which results from under-expression, over-expression or altered spatial or temporal expression of the gene. Individuals carrying mutations in the gene may be detected at the DNA level by a variety of techniques well known in the art.

Nucleic acids for diagnosis may be obtained from a subject's cells, such as from blood, urine, saliva, tissue biopsy or autopsy material. The genomic DNA may be used directly for detection or it may be amplified enzymatically by using PCR, preferably RT-PCR, or other amplification techniques prior to analysis. RNA or cDNA may also be used in similar fashion. Deletions and insertions can be detected by a change in size of the amplified product in comparison to the normal genotype. Point mutations can be identified by hybridizing amplified DNA to labeled mGRR nucleotide sequences. Perfectly matched sequences can be distinguished from mismatched duplexes by RNase digestion or by differences in melting temperatures.

DNA sequence difference may also be detected by alterations in the electrophoretic mobility of DNA fragments in gels, with or without denaturing agents, or by direct DNA sequencing (see, for instance, Myers et al, Science (1985) 230:1242). Sequence changes at specific locations may also be revealed by nuclease protection assays, such as RNase and S 1 protection or the chemical cleavage method (see Cotton et al., Proc Natl Acad Sci USA (1985) 85: 4397-4401).

An array of oligonucleotides probes comprising the mGRR polynucleotide sequence or fragments thereof can be constructed to conduct efficient screening of e.g., genetic mutations. Such arrays are preferably high density arrays or grids. Array technology methods are well known and have general applicability and can be used to address a variety of questions in molecular genetics including gene expression, genetic linkage, and genetic variability, see, for example, M.Chee et al., Science, 274, 610-613 (1996) and other references cited therein.

Detection of abnormally decreased or increased levels of polypeptide or mRNA expression may also be used for diagnosing or determining susceptibility of a subject to a disease of the invention. Decreased or increased expression can be measured at the RNA level using any of the methods well known in the art for the quantitation of polynucleotides, such as, for example, nucleic acid amplification, for instance PCR, RT-PCR, RNase protection, Northern blotting and other hybridization methods. Assay techniques that can be used to determine levels of a protein, such as a polypeptide of the present invention, in a sample derived from a host are well-known to those skilled in the art. Such assay methods include radioimmunoassays, competitive-binding assays, Western Blot analysis and ELISA assays.

Thus in another aspect, the present invention relates to a diagnostic kit comprising:
(a) a polynucleotide of the present invention, preferably the nucleotide sequence of SEQ ID NO: 1, SEQ ID NO: 3 or SEQ ID NO: 5, or a fragment or an RNA transcript thereof;
(b) a nucleotide sequence complementary to that of (a);
(c) a polypeptide of the present invention, preferably the polypeptide of SEQ ID NO: 2, SEQ ID NO: 4 or SEQ ID NO: 6 or a fragment thereof; or
(d) an antibody to a polypeptide of the present invention, preferably to the polypeptide of SEQ ID NO: 2, SEQ ID NO: 4 or SEQ ID NO: 6.

It will be appreciated that in any such kit, (a), (b), (c) or (d) may comprise a substantial component. Such a kit will be of use in diagnosing a disease or susceptibility to a disease, particularly diseases of the invention, amongst others.

The polynucleotide sequences of the present invention are valuable for chromosome localisation studies. The sequence is specifically targeted to, and can hybridize with, a particular location on an individual human chromosome. The mapping of relevant sequences to chromosomes according to the present invention is an important first step in correlating those sequences with gene associated disease. Once a sequence has been mapped to a precise chromosomal location, the physical position of the sequence on the chromosome can be correlated with genetic map data. Such data are found in, for example, V. McKusick, Mendelian Inheritance in Man (available on-line through Johns Hopkins University Welch Medical Library). The relationship between genes and diseases that have been mapped to the same chromosomal region are then identified through linkage analysis (co-inheritance of physically adjacent genes). Precise human chromosomal localisations for a genomic sequence (gene fragment etc.) can be determined using Radiation Hybrid (RH) Mapping (Walter, M. et al., (1994) A method for constructing radiation hybrid maps of whole genomes, Nature Genetics 7, 22-28). A number of RH panels are available from Research Genetics (Huntsville, Ala., USA) e.g. the—GeneBridge4 RH panel (Gyapay G et al., Hum Mol. Genet. 1996 March; 5(3): 339-46). To determine the chromosomal location of a gene using this panel, 93 PCRs are performed using primers designed from the gene of interest on RH IDNAs. Each of these DNAs contains random human genomic fragments maintained in a hamster background (human/hamster hybrid cell lines). These PCRs result in 93 scores indicating the presence or absence of the PCR product of the gene of interest. These scores are compared with scores created using PCR products from genomic sequences of known location. This comparison is conducted at hftp://www.genome.wi.mit.edu/.

The polynucleotide sequences of the present invention are also valuable tools for tissue expression studies. Such studies allow the determination of expression patterns of polynucleotides of the present invention that may give an indication as to the expression patterns of the encoded polypeptides in tissues, by detecting the mRNAs that encode them. The techniques used are well known in the art and include in situ hydridisation techniques to clones arrayed on a grid, such as cDNA microarray hybridisation (Schena et al, Science, 270, 467-470, 1995 and Shalon et al., Genome Res, 6, 639-645, 1996) and nucleotide amplification techniques such as PCR. A preferred method uses the TAQMAN™ technology available from Perkin Elmer. Results from these studies can provide an indication of the normal function of the polypeptide in the organism. In addition, comparative studies of the normal expression pattern of mRNAs with that of mRNAs encoded by an alternative form of the same gene (for example, one having an alteration in polypeptide coding potential or a regulatory mutation) can provide valuable insights into the role of the polypeptides of the present invention, or that of inappropriate expression thereof in disease. Such inappropriate expression may be of a temporal, spatial or simply quantitative nature.

The chromosomal localization can also be inferred using public domain databases, for example ENSEMBL (http://www.ensembl.orq/). The human mGRR1 gene maps on human Chromosome 10 p11.2-p12, the human mGRR2 gene maps on human chromosome Chr 17q11.1.

A further aspect of the present invention relates to antibodies. The polypeptides of the invention or their fragments, or cells expressing them, can be used as immunogens to produce antibodies that are immunospecific for polypeptides of the present invention. The term “immunospecific” means that the antibodies have substantially greater affinity for the polypeptides of the invention than their affinity for other related polypeptides in the prior art.

Antibodies generated against polypeptides of the present invention may be obtained by administering the polypeptides or epitope-bearing fragments, or cells to an animal, preferably a non-human animal, using routine protocols. For preparation of monoclonal antibodies, any technique which provides antibodies produced by continuous cell line cultures can be used. Examples include the hybridoma technique (Kohler, G. and Milstein, C., Nature (1975) 256:495-497), the trioma technique, the human B-cell hybridoma technique (Kozbor et aL, Immunology Today (1983) 4:72) and the EBV-hybridoma technique (Cole et aL, Monoclonal Antibodies and Cancer Therapy, 77-96, Alan R. Liss, Inc., 1985).

Techniques for the production of single chain antibodies, such as those described in U.S. Pat. No. 4,946,778, can also be adapted to produce single chain antibodies to polypeptides of this invention. Also, transgenic mice, or other organisms, including other mammals, may be used to express humanized antibodies.

The above-described antibodies may be employed to isolate or to identify clones expressing the polypeptide or to purify the polypeptides by affinity chromatography. Antibodies against polypeptides of the present invention may also be employed to treat diseases of the invention, amongst others.

Polypeptides and polynucleotides of the present invention may also be used as vaccines. Accordingly, in a further aspect, the present invention relates to a method for inducing an immunological response in a mammal that comprises inoculating the mammal with a polypeptide of the present invention, adequate to produce antibody and/or T cell immune response, including, for example, cytokine-producing T cells or cytotoxic T cells, to protect said animal from disease, whether that disease is already established within the individual or not. An immunological response in a mammal may also be induced by a method comprises delivering a polypeptide of the present invention via a vector directing expression of the polynucleotide and coding for the polypeptide in vivo in order to induce such an immunological response to produce antibody to protect said animal from diseases of the invention. One way of administering the vector is by accelerating it into the desired cells as a coating on particles or otherwise. Such nucleic acid vector may comprise DNA, RNA, a modified nucleic acid, or a DNA/RNA hybrid. For use a vaccine, a polypeptide or a nucleic acid vector will be normally provided as a vaccine formulation (composition). The formulation may further comprise a suitable carrier. Since a polypeptide may be broken down-in the stomach, it is preferably administered parenterally (for instance, subcutaneous, intramuscular, intravenous, or intradermal injection). Formulations suitable for parenteral administration include aqueous and non-aqueous sterile injection solutions that may contain anti-oxidants, buffers, bacteriostats and solutes that render the formulation instonic with the blood of the recipient; and aqueous and non-aqueous sterile suspensions that may include suspending agents or thickening agents.

The formulations may be presented in unit-dose or multi-dose containers, for example, sealed ampoules and vials and may be stored in a freeze-dried condition requiring only the addition of the sterile liquid carrier immediately prior to use. The vaccine formulation may also include adjuvant systems for enhancing the immunogenicity of the formulation, such as oil-in water systems and other systems known in the art. The dosage will depend on the specific activity of the vaccine and can be readily determined by routine experimentation.

Polypeptides of the present invention have one or more biological functions that are of relevance in one or more disease states, in particular the diseases of the invention hereinbefore mentioned. It is therefore useful to identify compounds that stimulate or inhibit the function or level of the polypeptide. Accordingly, in a further aspect, the present invention provides for a method of screening compounds to identify those that stimulate or inhibit the function or level of the polypeptide. Such methods identify agonists or antagonists that may be employed for therapeutic and prophylactic purposes for such diseases of the invention as hereinbefore mentioned. Compounds may be identified from a variety of sources, for example, cells, cell-free preparations, chemical libraries, collections of chemical compounds, and natural product mixtures. Such agonists or antagonists so-identified may be natural or modified substrates, ligands, receptors, enzymes, etc., as the case may be, of the polypeptide; a structural or functional mimetic thereof (see Coligan et al., Current Protocols in Immunology 1(2): Chapter 5 (1991)) or a small molecule.

The screening method may simply measure the binding of a candidate compound to the polypeptide, or to cells or membranes bearing the polypeptide, or a fusion protein thereof, by means of a label directly or indirectly associated with the candidate compound. Alternatively, the screening method may involve measuring or detecting (qualitatively or quantitatively) the competitive binding of a candidate compound to the polypeptide against a labelled competitor (e.g. agonist or antagonist). Further, these screening methods may test whether the candidate compound results in a signal generated by activation or inhibition of the polypeptide, using detection systems appropriate to the cells bearing the polypeptide. Inhibitors of activation are generally assayed in the presence of a known agonist and the effect on activation by the agonist by the presence of the candidate compound is observed. Further, the screening methods may simply comprise the steps of mixing a candidate compound with a solution containing a polypeptide of the present invention, to form a mixture, measuring a HGRL101 activity in the mixture, and comparing the HGRL101 activity of the mixture to a control mixture which contains no candidate compound.

Polypeptides of the present invention may be employed in conventional low capacity screening methods and also in high-throughput screening (HTS) formats. Such HTS formats include not only the well-established use of 96- and, more recently, 384-well micotiter plates but also emerging methods such as the nanowell method described by Schullek et al, Anal Biochem., 246, 20-29, (1997).

Fusion proteins, such as those made from Fc portion and mGRR polypeptide, as hereinbefore described, can also be used for high-throughput screening assays to identify antagonists for the polypeptide of the present invention (see D. Bennett et aL, J Mol Recognition, 8:52-58 (1995); and K. Johanson et al., J Biol Chem, 270(16):9459-9471 (1995)).

The polynucleotides, polypeptides and antibodies to the polypeptide of the present invention may also be used to configure screening methods for detecting the effect of added compounds on the production of mRNA and polypeptide in cells. For example, an ELISA assay may be constructed for measuring secreted or cell associated levels of polypeptide using monoclonal and polyclonal antibodies by standard methods known in the art. This can be used to discover agents that may inhibit or enhance the production of polypeptide (also called antagonist or agonist, respectively) from suitably manipulated cells or tissues.

A polypeptide of the present invention may be used to identify membrane bound or soluble receptors, if any, through standard receptor binding techniques known in the art. These include, but are not limited to, ligand binding and crosslinking assays in which the polypeptide is labeled with a radioactive isotope (for instance, ¹²⁵I), chemically modified (for instance, biotinylated), or fused to a peptide sequence suitable for detection or purification, and incubated with a source of the putative receptor (cells, cell membranes, cell supernatants, tissue extracts, bodily fluids). Other methods include biophysical techniques such as surface plasmon resonance and spectroscopy. These screening methods may also be used to identify agonists and antagonists of the polypeptide that compete with the binding of the polypeptide to its receptors, if any. Standard methods for conducting such assays are well understood in the art.

Examples of antagonists of polypeptides of the present invention include antibodies or, in some cases, oligonucleotides or proteins that are closely related to the ligands, substrates, receptors, enzymes, etc., as the case may be, of the polypeptide, e.g., a fragment of the ligands, substrates, receptors, enzymes, etc.; or a small molecule that bind to the polypeptide of the present invention but do not elicit a response, so that the activity of the polypeptide is prevented.

Screening methods may also involve the use of transgenic technology and the mGRR gene. The art of constructing transgenic animals is well established. For example, the mGRR gene may be introduced through microinjection into the male pronucleus of fertilized oocytes, retroviral transfer into pre- or post-implantation embryos, or injection of genetically modified, such as by electroporation, embryonic stem cells into host blastocysts. Particularly useful transgenic animals are so-called “knock-in” animals in which an animal gene is replaced by the human equivalent within the genome of that animal. Knock-in transgenic animals are useful in the drug discovery process, for target validation, where the compound is specific for the human target. Other useful transgenic animals are so-called “knock-out” animals in which the expression of the animal ortholog of a polypeptide of the present invention and encoded by an endogenous DNA sequence in a cell is partially or completely annulled. The gene knock-out may be targeted to specific cells or tissues, may occur only in certain cells or tissues as a consequence of the limitations of the technology, or may occur in all, or substantially all, cells in the animal. Transgenic animal technology also offers a whole animal expression-cloning system in which introduced genes are expressed to give large amounts of polypeptides of the present invention.

Screening kits for use in the above described methods form a further aspect of the present invention. Such screening kits comprise:
(a) a polypeptide of the present invention;
(b) a recombinant cell expressing a polypeptide of the present invention,
(c) a cell membrane expressing a polypeptide of the present invention; or
(d) an antibody to a polypeptide of the present invention; which polypeptide is preferably that of SEQ ID NO: 2, SEQ ID NO: 4 or SEQ ID NO: 6.

It will be appreciated that in any such kit, (a), (b), (c) or (d) may comprise a substantial component.

All publications and references, including but not limited to patents and patent applications, cited in this specification are herein incorporated by reference in their entirety as if each individual publication or reference were specifically and individually indicated to be incorporated by reference herein as being fully set forth. Any patent application to which this application claims priority is also incorporated by reference herein in its entirety in the manner described above for publications and references.

The following examples illustrate the invention.

### EXAMPLES

### Example 1

### Cloning of Human mGRR1a and Human mGRR1b

Using the amino acid sequence of GABA-B receptors (accessions Y10370, AJ011318) as queries in a tblastn search of the Celera human genomic database a sequence was identified (GA_{—}15234422) with limited similarity to putative transmembrane domains of GABA-B and metabotropic glutamate receptors.

To clone the corresponding cDNA 5′- and 3′-RACE reactions are performed using Clontech Marathon RACE cDNA (human brain, cat. no. 7400-1, BD Clontech, Basel, Switzerland ) essentially as described by the manufacturer. cDNA is transcribed from poly A(+) RNA (total human brain) purchased from Clontech (cat. no. 6543-1). The GIBCO-BRL cDNA synthesis module (Life Technologies, Basel, Switzerland) is used according to the manufacturer's instruction. Single-stranded cDNAs are synthesized using both oligo(dT) and random primers in separate reactions (2.5 μg of each RNA, 20 μl ). Before use in PCR 10 mM Tris, 1 mM EDTA pH 8.5 (TE) is added to the cDNA synthesis reactions to a final volume of 100 μl. PCRs are carried out on a MWG Primus cycler. Equal volumes from oligo (dT) and random primer cDNA synthesis reactions are combined. 1 μl cDNA mixture is used in a 50 μl PCR reaction (Sambrook et al., Molecular Cloning: A Laboratory Manual, 2nd Ed., Cold Spring Harbor Laboratory Press, Cold Spring Harbor, N.Y. (1989)).

RACE primers designed from sequence GA_{—}15234422 are: 5′-RACE: 5′-GCG GGG CTC ATG GAA TGC CGA TGG GAC-3′ (SEQ ID NO: 9) and 3′-RACE: 5′-GTC CCA TCG GCA TTC CAT GAG CCC CGC-3′ (SEQ ID NO: 10). 35 cycles are performed on a MWG Biotech cycler (initial denaturation at 95° C. for 3, 95° C. for 30 seconds (denaturation), 72° C. for 4 min (annealing and extension)). Advantage polymerase as outlined in the Clontech Marathon RACE kit description is used. A second PCR amplification step is performed using a nested gene-specific primer set (5′-RACE: 5′-CCG CAC TGC ATA GCA GAG ATA AAC ACC-3′ (SEQ ID NO: 7) and 3′-RACE: 5′-CAA TGA GCT CAT CAT CTC TGC TAT ATT CC-3′ (SEQ ID NO: 8)) that are used together with the AP2 primers supplied with the Marathon cDNA kit. 30 PCR cycles are performed using the conditions as above. RACE clones are subcloned into pCRII-topo (Invitrogen, Groningen, The Netherlands) and sequenced. By blastn searches of public domain databases (genbank) part of the 3′-RACE sequences are observed to be identical to a previously published sequence, KIAA1136 (accession AB032962). This sequence has been submitted to genbank as a partial cDNA sequence of unknown function cloned from brain.

The sequence information obtained from RACE clones is used to design primer sequences for cloning of the entire open reading frames. The PCR primers used for mGRR1a are: 5′-CAC CAT GGC TTA CCC CTT ACT CCT CTG C-3′ (SEQ ID NO: 11) and 5-CGT TGT TGC TCT TGC CCC CCT GGT CCT C-3′ (SEQ ID NO: 12). The 5′-primer containes upstream the putative start codon the minimal Kozak consensus sequence CACC (Kozak, Nucl Acid Res., 1987, 15., 8125-8132). The primers for mGRR1b are: 5′-GTG GGA CCA GCT GTG CTG CCA TTG ATC-3′ (SEQ ID NO: 13) and 5′-CGT TGT TGC TCT TGC CCC CCT GGT CCT C-3′ (SEQ ID NO: 14). Using 1 μl total human brain cDNA mixture (as described above) as template 35 PCR cycles are performed at: initial denaturation at 95° C. for 3, 95° C. for 30 seconds (denaturation), 65° C. for 30 seconds, 72° C. for 8 min. Proofreading Pfu polymerase from Promega (Wallisellen, Switzerland) is used. The cDNAs are sequenced (SEQ ID NO: 1, SEQ ID NO: 3) and inserted into a mammalian expression vector (pcDNA3.1-topo, Invitrogen).

One polypeptide of the present inventions (mGRR1a, corresponding DNA sequence SEQ ID NO: 1) contains an open reading frame for a protein of 1212 amino acids (SEQ ID NO: 2). A putative signal peptide sequence (MAYPLLLCLLLAQLGLG (SEQ ID NO: 15)) is located at the very N term of SEQ ID NO: 2 suggesting an extracellular location of the N terminal protein sequence. A sequence corresponding to an N-terminal splice variant of mGRR1; termed mGRR1b (SEQ ID NO: 4), contains an open reading frame of 1183 amino acids (SEQ ID NO: 5). The deduced protein sequences have the structural features characteristic of family 3 G protein-coupled receptors: 1) Hydrophobicity plots predict a protein with 7 putative transmembrane domains. 2) Similarity to family 3 GPCRs is substantiated by blastp searches (local alignments of putative transmembrane regions): 21% identical and 45% similar residues are found for mGRR1a compared to drosophila melanogaster metabotropic glutamate receptor (accession P91685); 22% identical and 43% similar residues are found for mGRR1a compared to putative drosophila melanogaster putative metabotropic GABA-B receptor subtype 3 (accession AF318274, local alignments of putative transmembrane regions). Closest homolog to mGRR1a in the drosophila genome is putative gene product CG11923 (flybase accession FBgn0031642) with 27% identical and 43% similar residues (blastp local alignment residues 241-745 of SEQ ID NO: 2 with residues 109-588 of CG11923. The function of CG11923 has been classified as G protein linked receptor. Closest mammalian GPCR homolog in public domain databases is metabotropic glutamate receptor type 3 (accession Q14832) with 23% identical and 41% similar residues (local alignment residues 385 to 640 of SEQ ID NO: 2 with residues 540-801 of mGluR3). Similarity of mGRR to family 3 GPCRs is restricted to the putative transmembrane spanning regions. No significant similarity to any known proteins is found for the N-terminal putatively extracellular domain as well as for the C-terminal putatively intracellular domain of mGRR.

TABLE 2
Putative transmembrane regions of mGRR1a.

| Transmembrane | | |
|---|---|---|
| domain | Position (SEQ ID 2) | Length |
| I | 415-437 | 23 |
| II | 447-469 | 23 |
| III | 483-505 | 23 |
| IV | 523-545 | 23 |
| V | 580-602 | 23 |
| VI | 618-640 | 23 |
| VII | 647-669 | 23 |

### Example 2

### Cloning of Human mGRR2

Using the amino acid sequence of GABA-B receptors (accessions Y10370, AJ011318) as queries in a tblastn search of the Celera human genomic database a sequence was identified (GA_{—}15091955) with limited similarity to putative transmembrane domains of GABA-B and metabotropic glutamate receptors.

To clone the corresponding cDNA 5′- and 3′-RACE reactions are performed using Clontech Marathon RACE cDNA (human brain, cat. no. 7400-1, BD Clontech, Basel, Switzerland ) essentially as described by the manufacturer. cDNA is transcribed from poly A(+) RNA (total human brain) purchased from Clontech (cat. no. 6543-1). The GIBCO-BRL cDNA synthesis module (Life Technologies, Basel, Switzerland) is used according to the manufacturer's instruction. Single-stranded cDNAs are synthesized using both oligo(dT) and random primers in separate reactions (2.5 μg of each RNA, 20 μl ). Before use in PCR 10 mM Tris, 1 mM EDTA pH 8.5 (TE) is added to the cDNA synthesis reactions to a final volume of 100 μl. PCRs are carried out on a MWG Primus cycler. Equal volumes from oligo (dT) and random primer cDNA synthesis reactions are combined. 1 μl cDNA mixture is used in a 50 μl PCR reaction (Sambrook et al., Molecular Cloning: A Laboratory Manual, 2nd Ed., Cold Spring Harbor Laboratory Press, Cold Spring Harbor, N.Y. (1989)).

RACE primers designed from sequence GA_{—}15091955 are: 5′-RACE: 5′-CCC GCT GCT CAG AAG GGC ACT CCG CTG G-3′ (SEQ ID NO: 24) and 3′-RACE: 5′-TGG ACC GTG GGC GCC CTG GAG CGA GGC-3′ (SEQ ID NO: 25). 35 cycles are performed on a MWG Biotech cycler (initial denaturation at 95° C. for 3, 95° C. for 30 seconds (denaturation), 72° C. for 4 min (annealing and extension)). Experimental procedures as described in in the Clontech Marathon RACE kit description are used. A second PCR amplification step is performed using a nested gene-specific primer set (5′-RACE: 5′-GGG CCG TTC GAG ACA GAA ACA GCT GCA G-3′ (SEQ ID NO: 27) and 3′-RACE: 5′-GCT GGG ACT ACA TCA TGG TTG TGG CTG-3′ (SEQ ID NO: 26) that are used together with the AP2 primers supplied with the Marathon cDNA kit. 30 PCR cycles are performed using the conditions as above. RACE clones are subcloned into pCRII-topo (Invitrogen, Groningen, The Netherlands) and sequenced.

The sequence information obtained from RACE clones is used to identify corresponding genomic sequences and the mGRR2 gene locus on human Chr 17q11.1 (http://www.ncbi.nlm.nih.gov/qenome/quide/human). Transcribed sequences are predicted by the genscan program (http://qenes.mit.edu/GENSCAN.html) and PCR primers are designed covering the putative open reading frame. The primer pairs are: 5′-CAC CGC CTC TGC CTG GGC TCT CCT G-3′ (F1) (SEQ ID NO: 28) and 5′-CGC TGC TCA GAA GGG CAC TCC GCT GG-3′ (SEQ ID NO: 29); 5′-GGA TTC CTG CTG CTT TAC TTT CCT GTC-3′ (SEQ ID NO: 30) and 5′-CCA CAG ACA CAC TTC AGC AAT GCC-3′ (SEQ ID NO: 31); 5′-CCA GGA AGG TGG AGA AGC CTG GGT GGG-3′ (SEQ ID NO: 32) and 5′-CTA ACT TGC CCT GTA GCA CTC CTC-3′ (R1) (SEQ ID NO: 33). Using 1 μl total human brain cDNA mixture (as described above) as template 35 PCR cycles are performed at: initial denaturation at 95° C. for 3, 95° C. for 30 seconds (denaturation), 62-68° C. for 30 seconds, 72° C. for 8 min. Proofreading Pfu polymerase from Promega (Wallisellen, Switzerland) is used. The full length sequence is assembled by splicing through overlap extension (Methods Enzymol. 1993; 217:270-279) in a PCR reaction using Pfu polymerase and primers F1 and R1 as above (25 PCR cycles are performed at: initial denaturation at 95° C. for 3, 95° C. for 30 seconds (denaturation), 68° C. for 30 seconds, 72° C. for 14 min) and the cDNA sequences inserted into a mammalian expression vector (pcDNA3.1-zeo, Invitrogen).

One polypeptide of the present inventions (mGRR2, corresponding DNA sequence SEQ ID NO: 5) contains an open reading frame for a protein of 2367 amino acids (SEQ ID NO:6). A putative signal peptide sequence MGTRGAVMPPPMWGLLGCCFVCAWALG (SEQ ID NO: 34) is located at the very N term of mGRR2 polypeptide (SEQ ID NO: 6) suggesting an extracellular location of the N terminal protein sequence. The amino acid sequence of hmGRR2 is 51% identical to mGRRa with 58% similar residues (bestfit alignment of amino acid residues 81 to 770 of mGRRa with residues 61 to 737 of mGRR2).

Expression profiling by PCR using cDNAs derived from different brain regions (Clontech) and RACE primers as above reveals reveal a widespread distribution of the mGRR2 mRNA in brain with an overlapping expression profile compared to mGRR1a/b.

### Example 3

### Cloning of Partial Rat mGRR1

A rat mGRR1 cDNA clone is identified in a arrayed dorsal root ganglion cDNA library (Novartis origin) using the human mGRR1a sequence (SEQ ID NO: 1) as query in a blastn search. Plasmid DNA from this clone is isolated and the cDNA insert partially sequenced (SEQ ID NO: 16); the nucleotide sequence is 80% identical to the corresponding human mGRR1 and b sequence (SEQ ID NO: 1, SEQ ID NO: 3).

### Example 4

### Mammalian Cell Expression (In Situ Hybridisation)

As templates for the synthesis of ³²P-UTP sense and antisense riboprobes fragments of the rat mGRR cDNA (SEQ ID NO: 16) are ampified by PCR using primers with attached T7 promotor sequences. The primer sequences are 5′-AAT GGA AGT CAG TTG TAC AC-3′ (SEQ ID NO: 17) and 5′-TTA TAC ACT CAC TAT AGG GAA ATG TCC CTT TAA CAG GCT G-3′ (SEQ ID NO: 18) (antisense template) and 5′-TAA TAC GAC TCA CTA TAG GGA AAT GGA AGT CAG TTG TAC AC-3′ (SEQ ID NO: 19) and 5′-AAT GTC CCT TTA ACA GGC TG-3′ (SEQ ID NO: 20) (sense template). 25 PCR cycles are performed on a MWG primus cycler using 50 ng of template plasmid (rat mGRR, SEQ ID NO: 16). PCR conditions are 95° C. for 30 seconds (denaturation), 68° C. for 1 min (annealing), and 72° C. for 1 min (extension). PCR products of the expected size (590 bp) are obtained, purified through spin columns (Roche Diagnostics, Indianapolis, USA, cat no. 1 732 668) and used for the synthesis of of ³⁵S-labeled RNA probes.

Probe synthesis is carried out with an RNA transcription kit (Stratagene, La Jolla, Calif., USA) as follows: 6 μl of [α-³⁵S]UTP and 6 μl of [α-³⁵S]ATP (specific activity 1200 Ci/mmol, NEN, Boston, Mass., USA) are evaporated in a 1.5 ml tube. Afterwards 2 μl 5× transcription buffer, 1 μl 100 mM DTT, 3 μl DEPC-H₂O, 1 μl of a solution containing 10 μl 100 mM CTP, 10 μl 100 mM GTP, 30 μl DEPC-H₂O), 1 μl RNasin, 1 μl of T7 or T3 polymerase and 200 ng of PCR product is added. After 1-1.5 h of incubation at 37° C., the reaction is stopped by adding 90 μl of a solution containing 50 μl SDS 20%, 100 μl 0.1 M DDT and 850 μl of 10 mM Tris-1 mM EDTA pH 7.4). After purification on a sephadex G-50 spin-column (Boehringer, Mannheim, Germany), the radioactivity is measured by scintillation counting. The hybridization mixture is prepared from two separate solutions: Solution A contained 10 ml formamide, 4 ml 50% dextran sulfate, 400 μl 50× Denhard's solution (5 g ficoll, 5 g polyvinylpyrolidone and 5 g bovine serum albumine in 500 ml H₂O), 40 μl 0.5 M EDTA pH 8.0, 200 μl 1 M Tris pH 8.0 and 1.2 ml 5 M NaCl; Solution B is composed of 1-5 μl of the ³⁵S-labeled probe (the exact volume is defined as to reach a concentration of 10⁷ cpm/ml in the final medium), 100 μl tRNA, 100 μl 0.1 M DTT, completed with DEPC-H₂O to 2 ml and heated at 65° C. for 5 min. The final hybridization mixture is made up with 8 ml of solution A and 2 ml of solution B. well mixed, syringe filtered, heated again 5 min at 65° C. and centrifuged for 5 min at 10′000 g to remove air bubbles.

Rat brains are cut into 8 to 16 μm thick coronal sections on a cryostat at −20 to −25° C. and mounted onto a gelatine-poly-L-lysine-coated slides. The sections are vacuum-dryed overnight at room temperature, fixed for 5 min in 4% (w/v) ice-cold paraformaldehyde, washed 3 times 1 min in 1×PBS. They are either used the same day for hybridization or stored in sealed slide boxes at −70° C.

On the day of the hybridization, the frozen slide boxes are kept closed until room temperature is reached. Then, the slides are dipped succesively into staining dishes containing 250 ml 0.3 M triethanolamine, acetylated with the same triethanolamine added with 625 μl acetic anhydride, dehydrated in graded ethanol (50, 70, 95, 100, 100%) and vacuum-dryed for 1-3 h. 75 μl of the hybridization mixture is pipetted on a coverslip. At the contact with the glass slide, the solution spreads uniformly by capillarity all over the sections. Once sealed with DPX, the slides are placed and kept at 56° C. for 16-20 h. The slides are then cooled at room temperature, the hardened DPX is removed and the slides are dipped into a 4×SSC buffer for 20 min or more until the coverslips came off. The high stringency wash in 0.1×SSC is at 68° C. For emulsion-dipping, the sections are defatted 5 min in 95% ethanol, 3 times 5 min in 100% ethanol, once 5 min in xylene, once 30 min and 3 times again in 100% ethanol (same solution as before), and vacuum-dryed for at least 1 h. Then, in the dark-room, the liquid nuclear emulsion (Kodak, NTB2, Integra Biosciences AG, Wallisellen, Switzerland, state/country) is diluted 1:1 in distilled water pre-heated at 52° C. The mixture is allowed to dissolve for 15 min in a 52° C. water bath. Then, solution is very gently agitated by 180° rotations to mix well but avoiding bubble formation, rested for another 15 min. The homogeneous mixture is poured into a special dipping flask and kept again for 15 min to let the bubble come off. Then, the slides are dipped once into the emulsion and dried on a holder in a dark chamber for 3 h. The slides are stored in sealed slide-boxes at 4° C. in the dark-room. After 16 to 60d exposure, the slides are proceeded for development. The developer D-19 and fixer (Kodak) used at standard concentration are cooled to 15° C. in ice. The slides are then dipped into to developer for 3.5 min, washed in 15° C. water for 15 sec and fixed for 6 min. Finally; they are washed in demineralized water for 1 h. The sections are counterstained or the slides are directly mounted for microscopic examination with 3 drops of histological mounting medium (Permount, Fisher Scientific, Pittsburgh, Pa., USA) before placing a coverslip and dried in slide-boxes for several days until the coverslips strongly adhered.

The observed brain specific regional distribution of mGRR mRNA provides information on possible indications for mGRR ligands. mGRR ligands are the natural ligand as well as modulators of mGRR activity, such as anti-mGRR antibodies and/or small molecules that agonize or antagonize mGRR-mediated signalling. Family 3 GPCRs such as metabotropic glutamate receptors and GABA-B receptors modulate synaptic transmission and in comparison to ion channel their effects are rather long-lasting and modulatory. The mRNA expression pattern in the brain of mGRR provides an indication that mGRR interacting molecules will have utility for treating neurological and/or psychiatric diseases, including but not limited to dementia, schizophrenia, depression, affective disorders, epillepsy, and motoric disorders.

The polypeptides of the present invention are expressed in all major brain structures (Table 1).

TABLE 1
Distribution of mRNA coding for mGRR1 throughout the rat brain.

| Brain areas, nuclei or cell types | | Expression level |
|---|---|---|
| Main olfactory bulb | | |
| | Periglomerular cells | + |
| | Mitral cells | +++ |
| | Granule cells | + |
| | Tufted cells | |

| | |
|---|---|
| Internal | ++ |
| External | +++ |

| Basal forebrain | | |
|---|---|---|
| | Caudate putamen | +++ |
| | Globus pallidus | 0 |
| | Nucleus Accumbens | ++ |
| | Olfactory Tubercle | ++ |
| | Islands of Calleja | 0 |
| | Ventral pallidum | ++ |
| Amygdaloid nuclei | | |
| | Basolateral nuclei | + |

Anterior
Posterior

| | |
|---|---|
| Basomedial nuclei | ++ |
| Central nucleus, lateral | + |
| Medial nucleus | + |

Posteroventral
Posterodorsal

| | |
|---|---|
| Lateral nucleus | +++ |

Ventromedian/ventrolateral
Dorsolateral

| | Posteromedial cortical amyg nuc. | ++ |
|---|---|---|
| Septal area | | |
| | Lateral septum | ++ |
| | Medial septum | |
| | Bed Nucleus Stria terminalis | |
| Hippocampal formation | | |
| | Pyramidal layer CA1 | ++ |
| | Pyramidal layer CA3 | +++ |
| | Dentate gyrus, granular layer | ++ |
| | Hilar cells | ++++ |
| | Other nonprincipal cells | +++ |
| | Subiculum dorsal | + |
| Thalamus | | |
| Dorsal Thalamus | | |
| | Anterior group | |

| | |
|---|---|
| Laterodorsal, ventrolateral (LDVL) | ++ |
| Laterodorsal, dorsomedial (LDDM) | ++ |

| | |
|---|---|
| Dorsal group (IMD) | ++ |
| Lateral group | |

| | |
|---|---|
| Lateral posterior mediorostral (LPMR) | ++ |

Medial group

| | |
|---|---|
| Paraventricular (PVP) | + |
| Mediodorsal (MDM), medial | +++ |
| Mediodorsal (MDC), centrolateral | +++ |

Ventral group

| | |
|---|---|
| Ventral medial (VM) | +++ |
| Ventral posteromedial (VPM) | + |
| Ventral posterolateral (VPL) | + |

| | |
|---|---|
| Posterior group | ++ |
| Reuniens | + |
| Geniculate group | |

| | Medial geniculate nuclei | |
|---|---|---|
| | Lateral geniculate nuclei, dorsal | ++ |
| | Lateral geniculate nuclei, ventral | ++ |
| Epithalamus | | |
| | Medial habenula | ++ |
| | Lateral habenula | ++ |
| Ventral thalamus | | |
| | Zona incerta | + |
| | Reticular thalamic nucleus | + |
| | Subthalamic nucleus | |

| Hypothalamus | | |
|---|---|---|
| | Median preoptic nucleus | + |
| | Periventricular nucleus | + |
| | Medial preoptic area | + |
| | Lateral preoptic area | + |
| | Anteroventral preoptic nucleus | + |
| | Anterior medial preoptic nucleus | + |
| | Nucleus of the diagonal band | + |
| | Magnocellular preoptic nucleus | + |
| | Arcuate nucleus | + |
| | Dorsomedial hypothalamic (DMD) | + |
| | Ventromedial hypothalamic | |

| | |
|---|---|
| Dorsomedial (VMHDM) | + |
| Ventrolateral (VMHVL) | + |

| | Supraoptic nucleus | + |
|---|---|---|
| | Mammillary bodies | ++ |
| | Paraventricular hypothalamic nucleus | + |
| Midbrain/Brainstem | | |
| | Substantia nigra | |

| | |
|---|---|
| Pars compacta | +++ |
| Pars reticulata | 0 |

| | |
|---|---|
| Ventral tegmental area | ++++ |
| Interpeduncular nucleus | + |
| Mesencephalic reticular nucleus | ++ |
| Superior colliculi | |

| | |
|---|---|
| Superficial gray layer | ++ |
| Optical layer | ++ |

| | Inferior colliculi | ++ |
|---|---|---|
| | Periacqueductal gray | + |
| | Ventral cochlear nucleus | + |
| | Locus coeruleus | +++ |
| | Raphe nucleus dorsalis | + |
| Pons-Medulla | | |
| | Pontine nucleus | +++ |
| | Mesencephalic trigeminal nucleus | +++ |
| | Central gray pontine | ++ |
| | Parabrachial nucleus | +++ |
| | Lateral superior olive | +++ |
| | Inferior olive | +++ |
| | Mesencephalic vestibular nucleus | ++ |
| | Nucleus solitary tract | ++ |
| Cortex | | |
| | Frontal | |

| | |
|---|---|
| Layer I | + |
| Layer II-III | ++ |
| Layer V | +++ |
| Layer VI | + |

Parietal

| | |
|---|---|
| Layer I | + |
| Layer II-III | ++ |
| Layer IV | ++++ |
| Layer V | +++ |
| Layer Via | + |
| Layer Vib | ++ |

| | |
|---|---|
| Piriform cortex | +++ |
| Indesium griseum | +++ |

| Cerebellum | | |
|---|---|---|
| | Molecular layer | + |
| | Granular layer | ++ |
| | Purkinje cells | 0 |
| | Golgi cells | ++++ |
| | Deep cerebellar nucleus (lateral) | + |
| White matter areas (glial cells) | | |
| | Anterior commissural | 0 |
| | Fornix | 0 |
| | Lateral olfactory tract | 0 |
| | Corpus callosum | 0 |

The hybridization probe used corresponds to C-terminal sequences that are common to mGRR1a and mGRR1b (pan probe). Levels of expression were estimated on coronal and sagittal brain section under darkfield microscope after having been exposed to nuclear emulsion for 10 days.
0 = no detectable expression;
+ = weak;
++ = moderate;
+++ =high;
++++ = very high.

The observed brain specific regional distribution of mGRR mRNA provides information on possible indications for mGRR ligands. mGRR ligands are the natural ligand as well as modulators of mGRR activity, such as anti-mGRR antibodies and/or small molecules that agonize or antagonize mGRR-mediated signaling. Family 3 GPCRs such as metabotropic glutamate receptors and GABA-B receptors modulate synaptic transmission and in comparison to ion channel their effects are rather long-lasting and modulatory.

### Example 5

### Immunoblot

Generation of HA tagged mGRR1a. A C-terminal hemagglutinin (HA) epitope (nucleotide sequence 5′-TATCCATATGATGTTCCAGATTATGCT (SEQ ID NO: 21) was added to the C-terminal sequence of mGRR1a. To this end, a PCR reaction was performed with the primers 5′-CAAGACTCCAGTTCTCCCAGAG (SEQ ID NO: 22) and 5′-TCTAGATCTAGACTAAGCATAATCTGGAACATCATATGGATACACTTTAAAACTATCCCAGATC (SEQ ID NO: 23) using human mGRR1a in pcDNA3.1 topo as template (50 ng). 25 PCR cycles were performed at 95° C. (30 sec), 68° C. (30 sec), 72° C. (30 sec). A PCR product of the expected size (417 bp) was obtained, double-digested with Bsgl and Xbal, gel purified (Qiaex, Qiagen) and used to replace the Bsgl/Xbal fragment of wild-type hmGRR1a in pcDNA3 topo. The construct was confirmed by sequencing of both strands.

Immunoblot. Membranes from transfected COS 1 cells were thawed, centrifuged and resuspended in HEPES buffer pH (125 mM NaCl, 5 mM KCl, 0.6 mM MgCl₂, 1.8 mM CaCl₂, 20 mM HEPES, 6 mM Dextrose; (Life Technologies #043-90174M)), 50 μg/ml Dnase I and diluted in sample buffer (125 mM Tris pH6.8, 1% SDS, 25 mM DTT, 5% glycerol/bromphenol blue). Proteins (20 μg per lane) were separated by SDS-PAGE using 4-15% gradient gels and electrophoretically transferred onto Immobilon-P PVDF membranes (Millipore AG, Volketswil, Switzerland). Non-specific binding was reduced by overnight incubation in NET-G buffer (150 mM NaCl, 50 mM Tris-Cl pH 7.4, 5 mM EDTA, 0.05% (v/v) Triton X100, 0.25% w/v gelatine). Subsequently the membranes were incubated with a monoclonal rat anti-HA-Peroxidase high affinity antibody (3F10; Roche cat no. 2013819, 1:500 in NET-G for 45 min at room temperature. After 3 washes with NET-G (10 min each) bound antibody was detected using the with enhanced chemiluminescent Western Blot reagents (Amersham Life Sciences #2106) and the membranes exposed to Biomax MR (Kodak) films.

Western blots of membranes from COS-1 cells after transfection with human GRR-HA in pcDNA3.1-topo (Gibco Life Sciences) shows a protein band at 134 kDa (as predicted from its protein sequence). The immunoblot indicates as it is done with cellular membranes that mGRR is found at the cellular membrane.

### Example 6

### Functional Analysis

mGRR1 a, mGRR1b and mGRR2may be co-expressed in recombinant expression systems such as HEK293 cells or COS cells together with putative interacting receptor proteins. Co-transfection of cDNA expression constructs may be for example done with the Effectene transfection agent (Qiagen). A functional read-out may involve analysis of agonist induced GTPyS binding such as described by Galvez et al., Mol. Pharmacol., 57, 419-426 (2000) or the activation of potassium channels (Lingenhoehl etal., Neuropharmacology, 38, 1667-1673 (1999)). Co-transfection of G proteins or chimeric G proteins may be used to generated a calcium signal (inositol phosphate accumulation) that may be measured as described (Galvez et al., EMBO J. 20, 2152-2159 (2001). Alternatively the binding of radiolabelled candidate ligands may be measured using membrane preparations derived from transfected cells. In addition, the following experiments may elucidate the function of mGRR1 and mGRR2:

a) Gene knockouts

The murine mGRR1 and mGRR2 genes are localized on proximal Chr 2 and distal Chr 11, respectively. This knowledge of the murine mGRR genes and their structure is used to design gene targeting constructs with the aim to generate mice in which the functions of mGRR1 and/or mGRR2 gene has been ablated. Knockout animals are generated by standard methods as desribed (Neuron. 2001; 31, 47-58) involving either constitutive or inducible knockouts. The phenotype of homozgous knockout mice is investigated in biochemical, pharmacological and electrophysiological paradigms in order to study a possible impairment of known biochemical and receptor pathways. The animals are also investigated in different behavioral paradigms (for example as described in (Neuron. 2001; 31, 47-58).

With CG11923 a putative drosophila homolog of mammalian mGRRs has been identified. The phenotype of drosophila flies in which the function of CG11923 has been discrupted by means of P element mutagenesis is investigated. The results are expected to shed light on the functions of mGRR1 and mGRR2 in vivo.

b) Interacting proteins

Using C or N terminal sequences as baits interacting proteins of mGRR1 and mGRR2 are identified. This involves yeast two hybrid screens of brain cDNA libraries as well as the generation of GST fusion proteins for subsequent pull down assays using brain extracts and mass spectoscropic analysis of proteins. The abovementioned methods are also used to investigate a possible heterodimeric interaction of mGRR1 and mGRR2. Potential interacting proteins are investigated in co-localization experiments in transfected cells and in neuronal cultures. In addition co-immunoprecipitation experiments are performed. At the very Cterm of human mGRR1a/b a a putative type 11 PDZ protein binding motif (J Biol Chem. 2002, 277, 15221-4) is identified suggesting that interaction of mGRR1a/b with known PDZ motif binding proteins such as PICK1 may direct mGRR to known GPCR signalling pathways (EMBO J. 2002, 21, 2990-9.) A putative interaction of human mGRR1 with PICK1 is investigated using co-localization and co-immunoprecipitation studies as described above.

c) Ligand fishing screen

A collection of putative GPCR ligands has been established in house. A bank of putative receptor ligands has been assembled for screening. The bank comprises: transmitters, hormones and chemokines; naturally occurring compounds which may be putative agonists for a human receptor, non-mammalian, biologically active peptides for which a mammalian counterpart has not yet been identified; and compounds not found in nature, but which activate receptors with unknown natural ligands. This bank is used to initially screen the receptor for known ligands, using both functional (i.e. calcium, cAMP, microphysiometer, oocyte electrophysiology, etc, see below) as well as binding assays.mGRR1 and mGRR2 are co-expressed in recombinant expression systems such as HEK293 cells or COS cells together with putative interacting receptor proteins (as above). This may also involve co-expression of mGRR1 and mGRR2. Co-transfection of cDNA expression constructs is for example done with the Effectene transfection agent (Qiagen). A functional read-out may involve analysis of agonist induced GTPyS binding such as described by Galvez et al., Mol. Pharmacol., 57, 419-426 (2000) or the activation of potassium channels (Lingenhoehl et al., Neuropharmacology, 38, 1667-1673 (1999)). Co-transfection of G proteins or chimeric G proteins are used to generated a calcium signal (inositol phosphate accumulation) that is measured as described (Galvez et al., EMBO J. 20, 2152-2159 (2001). Alternatively the binding of radiolabelled candidate ligands is measured using membrane preparations derived from transfected cells.

d) Extract/cell supernatant screening

A large number of mammalian receptors exist for which there remains, as yet, no cognate activating ligand (agonist). Thus, active ligands for these receptors may not be included within the ligands banks as identified to date.

Accordingly, the receptor of the invention is also functionally screened (using calcium, cAMP, microphysiometer, oocyte electrophysiology, etc., functional screens) against tissue extracts to identify natural ligands. Extracts that produce positive functional responses can be sequentially subfractionated until an activating ligand is isolated identified.

e) Antibodies and Peptides

Antibodies (polyclonal or monoclonal) are generated directed against N and C-terminal epitopes of mGRR. Peptides are designed that are expected to disrupt the association of mGRR proteins with putative interacting proteins as identified (such as PICK1 as above). Antibodies and or peptides are applied to cultured neuronal cells with the aim to disrupt the function of endogenously expressed mGRR proteins. The electrophysological properties of antibody and/or peptide treated cells are investigated in comparison to untreated cells.

### Example 7

### Ligand Binding Assays

Ligand binding assays provide a direct method for ascertaining receptor pharmacology and are adaptable to a high throughput format. The purified ligand for a receptor is radiolabeled to high specific activity (50-2000 Ci/mmol) for binding studies. A determination is then made that the process of radiolabeling does not diminish the activity of the ligand towards its receptor. Assay conditions for buffers, ions, pH and other modulators such as nucleotides are optimized to establish a workable signal to noise ratio for both membrane and whole cell receptor sources. For these assays, specific receptor binding is defined as total associated radioactivity minus the radioactivity measured in the presence of an excess of unlabeled competing ligand. Where possible, more than one competing ligand is used to define residual nonspecific binding.

mGRR1a, mGRR1b and mGRR2 may have the essentially the same ligands.

### Example 8

### Chromosomal Localization

The chromosomal localization is inferred using public domain databases, for example ENSEMBL (http://www.ensembl.orq/) using conventional techniques. The human mGRR maps on human Chr 10p11.2-p12.; the mGRR2 gene locus maps on human Chr 17q11.1.

1. An isolated mGRR polypeptide selected from one of the groups consisting of:
(a) an isolated mGRR polypeptide encoded by a polynucleotide comprising the sequence of SEQ ID NO: 3 or SEQ ID NO: 5;
(b) an isolated mGRR polypeptide comprising a polypeptide sequence having at least 96% identity to the polypeptide sequence of SEQ ID NO: 4 or SEQ ID NO: 6 and which shows properties in the ligand binding assay similar to those of mGRR;
(c) an isolated mGRR1 b or mGRR2 polypeptide comprising the polypeptide sequence of SEQ ID NO: 4 or SEQ ID NO: 6;
(d) an isolated mGRR polypeptide having at least 96% identity to the polypeptide sequence of SEQ ID NO: 4 or SEQ ID NO: 6 and which shows properties in the ligand binding assay similar to those of mGRR;
(e) the polypeptide sequence of SEQ ID NO: 4 or SEQ ID NO: 6;
(f) an isolated mGRR polypeptide having or comprising a polypeptide sequence that has an Identity Index of 0.96 compared to the polypeptide sequence of SEQ ID NO: 4 or SEQ ID NO: 6 and which shows properties in the ligand binding assay similar to those of mGRR; or
(g) a fragment or variant of such a polypeptide according to (a) to (f).

2. The isolated polypeptide as claimed in claim 1 which is the polypeptide sequence of SEQ ID NO: 4 or SEQ ID NO: 6.

3. An isolated mGRR polynucleotide selected from one of the groups consisting of:
(a) an isolated mGRR polynucleotide comprising a polynucleotide sequence having at least 80% identity to the polynucleotide sequence of SEQ ID NO: 3 or SEQ ID NO: 5 and which encodes for polypeptides which show properties in the ligand binding assay similar to mGRR;
(b) an isolated polynucleotide comprising the polynucleotide of SEQ ID NO: 3 or SEQ ID NO: 5;
(c) an isolated polynucleotide comprising at least 96% identity to the polynucleotide of SEQ ID NO: 3 or SEQ ID NO: 5 and which encodes for polypeptides which show properties in the ligand binding assay similar to mGRR;
(d) the isolated polynucleotide of SEQ ID NO: 3 or SEQ ID NO: 5;
(e) an isolated polynucleotide comprising a polynucleotide sequence encoding a polypeptide sequence having at least 96% identity to the polypeptide sequence of SEQ ID NO: 4 or SEQ ID NO: 6 and which encodes for polypeptides which show properties in the ligand binding assay similar to mGRR;
(f) an isolated polynucleotide comprising a polynucleotide sequence encoding the polypeptide of SEQ ID NO: 3 or SEQ ID NO: 5;
(g) an isolated polynucleotide comprising a polynucleotide sequence encoding a polypeptide sequence having at least 96% identity to the polypeptide sequence of SEQ ID NO: 4 or SEQ ID NO: 6 and which encodes for polypeptides which show properties in the ligand binding assay similar to mGRR;
(h) an isolated polynucleotide encoding the polypeptide of SEQ ID NO: 4 or SEQ ID NO: 6;
(i) an isolated polynucleotide comprising a polynucleotide sequence that has an Identity Index of 0.96 compared to the polynucleotide sequence of SEQ ID NO: 3 or SEQ ID NO: 5 and which encodes for polypeptides which show properties in the ligand binding assay similar to mGRR; or
k) an isolated polynucleotide comprising a polynucleotide sequence encoding a polypeptide sequence that has an Identity Index of 0.96 compared to the polypeptide sequence of SEQ ID NO: 4 or SED ID NO: 6 and which encode for polypeptides which show properties in the ligand binding assay similar to mGRR; or polynucleotides that are fragments or variants of the above mentioned polynucleotides or that are complementary to above mentioned polynucleotides, over the entire length thereof.

4. An isolated polynucleotide as claimed in claim 3 selected from the group consisting of:
(a) an isolated polynucleotide comprising the polynucleotide of SEQ ID NO: 3 or SEQ ID NO: 5;
(b) the isolated polynucleotide of SEQ ID NO: 3 or SEQ ID NO: 5;
(c) an isolated polynucleotide comprising a polynucleotide sequence encoding the polypeptide of SEQ ID NO: 4 or SEQ ID NO: 6; and
(d) an isolated polynucleotide encoding the polypeptide of SEQ ID NO: 4 or SEQ ID NO: 6.

5. An expression system comprising a polynucleotide capable of producing a polypeptide of claim 1 when said expression vector is present in a compatible host cell.

6. A recombinant host cell comprising the expression vector of claim 5 or a membrane thereof expressing the polypeptide of claim 1.

7. A process for producing a polypeptide of claim 1 comprising the step of culturing a host cell as defined in claim 6 under conditions sufficient for the production of said polypeptide and recovering the polypeptide from the culture medium.

8. A fusion protein consisting of the immunoglobulin Fc-region and any one polypeptide of claim 1.

9. An antibody immunospecific for the polypeptide of any one of claims 1 to 2.

10. A method of screening to identify compounds that stimulate or inhibit the function or level of the polypeptide of claim 1, comprising a method selected from the group consisting of:
(a) measuring or, detecting, quantitatively or qualitatively, the binding of a candidate compound to the polypeptide (or to the cells or membranes expressing the polypeptide) or a fusion protein thereof by means of a label directly or indirectly associated with the candidate compound;
(b) measuring the competition of binding of a candidate compound to the polypeptide (or to the cells or membranes expressing the polypeptide) or a fusion protein thereof in the presence of a labelled competitor;
(c) testing whether the candidate compound results in a signal generated by activation or inhibition of the polypeptide, using detection systems appropriate to the cells or cell membranes expressing the polypeptide;
(d) mixing a candidate compound with a solution containing a polypeptide of claim 1, to form a mixture, measuring activity of the polypeptide in the mixture, and comparing the activity of the mixture to a control mixture which contains no candidate compound; or
(e) detecting the effect of a candidate compound on the production of mRNA encoding said polypeptide or said polypeptide in cells, using for instance, an ELISA assay, and
(f) producing said compound according to biotechnological or chemical standard techniques.


SEQUENCE LISTING (34 sequences)

SEQ ID NO: 1 - DNA, 3660 bp, Homo sapiens
  CDS (1)..(3636): human mGRR1a
atggcttaccccttactcctctgcctcctgcttgctcagctgggattgggagctgttggcgccagccgcgacccccaaggacggccggattcccctcgagagaggaccccgaaggggaagccgcacgcccagcagccgggtcgagcctctgcctcggactcctcggctccctggagccgctccaccgatggcaccatcttggcgcagaaactcgccgaggaggtgcccatggacgtggcctcttacctctacaccggggactcccaccagctgaagcgagccaactgctccggccgctacgagttggcgggcctgccggggaagtggccagccctggccagcgcgcacccctccttgcaccgggcgctggacacactgacacacgccaccaacttcctcaacgtgatgctgcagagcaataagtcgcgggagcagaacttgcaggacgacctggattggtaccaggcgctggtgtggagccttctggagggcgagcccagcatctcccgggcggccatcaccttcagcaccgattcgctgtccgcaccggccccacaggtcttcctccaggccacgcgcgaggagagccgcatcctgctccaagacctgtcctcctccgcaccccacctggccaacgccactctggagaccgagtggttccacggcctccggcgcaagtggaggccccacttacaccgccgcggccccaatcaggggccccggggcctgggccacagctggcggcgcaaggacgggctcggcggggacaagagccacttcaagtggtctccgccttatctggagtgcgagaacgggagttacaagcccgggtggctggttactctttcctctgccatctacgggttgcagcctaacctggtcccggaattcaggggtgtcatgaaagttgacataaatcttcagaaagtggacattgaccaatgctcaagtgatggctggttttcaggaactcataaatgccacctcaacaattcagagtgtatgccaattaaaggcctaggattcgttcttggagcctatgagtgcatttgcaaagcaggattctatcatcctggagtcttaccagtgaacaactttcggagaaggggtccggatcagcatatttcaggaagtacaaaagatgtgtcagaagaagcctatgtctgcctaccttgcagggagggctgccccttctgtgctgatgacagcccatgcttcgtccaggaagataagtatttacgacttgccatcatctccttccaagccctgtgtatgctgctcgacttcgttagcatgctggtggtctaccactttcgcaaagcaaagagcatccgggcatcgggccttatcctgttggaaacgatcctttttggatctctgctcctatactttccagttgttattttgtactttgagccaagcacatttcgctgtattctcctaagatgggctcgtcttctcggttttgctactgtttacggaactgtcactctcaaacttcacagggttttgaaggtgtttctttcacgaacggctcaacgaattccatatatgactggcggacgggtcatgaggatgctggcagtaatactcttggtagtgttttggtttctcattggctggacttcatctgtgtgccagaatttggagaaacagatttcacttattggccaggggaaaacatccgatcacctcatcttcaatatgtgcctcattgaccgctgggactacatgacagcagttgctgaatttttattcctcttgtggggtgtttatctctgctatgcagtgcggacagtcccatcggcattccatgagccccgctatatggctgttgcagttcacaatgagctcatcatctctgctatattccatacaattagatttgttcttgcctcaagacttcagtctgattggatgttgatgctgtattttgcacatactcatttgactgtgacagtcaccattgggttgcttttgattccaaagttttcacattcaagcaataacccacgagatgatattgctacagaagcatatgaggatgagctagacatgggccgatctggatcctacctgaacagcagtatcaattcagcctggagtgagcacagcttggatccagaggacattcgggacgagctgaaaaaactctatgcccaactggaaatatataaaagaaagaagatgatcacaaacaacccccacctccagaaaaagcggtgctcgaagaagggcctaggtcgttccatcatgagacgcattacggagatcccagagacagtcagccggcagtgccctaaagaggacaaggagggcgccgaccatggcacagccaaaggcactgccctcatcaggaagaaccccccagagtcttcagggaacacagggaaatccaaggaggagaccctgaaaaaccgagtcttctcactcaagaaatcccacagcacttatgaccacgtgagagaccaaacggaagagtccagtagcctacccacagaaagccaagaggaggagacaacagaaaattccacactggaatccctgtcgggtaaaaaactaacacaaaaactaaaagaagacagcgaggctgagtccacggagtcggtgccgttggtgtgcaagtcagcaagcgctcacaacctcagctcagagaagaaaactgggcacccacgaacatcgatgttacagaagtctctcagtgtcatagcaagcgccaaggagaagactcttggattagctgggaaaacccaaacagcaggtgtggaagaacgcactaaatcccagaaacctttgccaaaagataaagagacaaacagaaatcactcaaattctgataacacagagactaaagatcctgccccccaaaactcaaatcctgcggaggagccaagaaagcctcagaaatctgggattatgaaacaacaaagggtcaaccccaccactgccaattctgacctgaacccaggcaccacccagatgaaggacaactttgacattggggaggtgtgtccttgggaggtttatgacctgacccctggtcctgtgccttcagaatcaaaagttcaaaagcacgtatctattgtggcttctgaaatggagaaaaaccccactttttccttaaaggagaaatctcaccacaagcctaaggcagctgaggtttgtcagcaatccaatcagaagcgcatagataaggctgaagtatgcctttgggagagccaaggccagtccattttggaagatgagaagcttttgatttccaagactccagttctcccagagagggcaaaagaggagaacggaggtcagcctcgtgcagccaatgtgtgtgctgggcagagcgaagaactgccccccaaagctgtagcatcaaaaacagagaatgaaaatctcaaccaaataggacaccaggaaaaaaagacatcttcttctgaggagaatgtgcgtggctcctataactcaagtaataacttccagcaacctttaacatcacgagcagaggtttgtccttgggagtttgagaccccagctcaaccaaatgctggaagaagtgtagctttacctgcctcttctgctctaagtgcaaataagatagcagggcctaggaaagaagagatctgggatagttttaaagtgtagcatctccaggaagaagaggaa

SEQ ID NO: 2 - protein, 1212 aa, Homo sapiens
MAYPLLLCLLLAQLGLGAVGASRDPQGRPDSPRERTPKGKPHAQQPGRASASDSSAPWSRSTDGTILAQKLAEEVPMDVASYLYTGDSHQLKRANCSGRYELAGLPGKWPALASAHPSLHRALDTLTHATNFLNVMLQSNKSREQNLQDDLDWYQALVWSLLEGEPSISRAAITFSTDSLSAPAPQVFLQATREESRILLQDLSSSAPHLANATLETEWFHGLRRKWRPHLHRRGPNQGPRGLGHSWRRKDGLGGDKSHFKWSPPYLECENGSYKPGWLVTLSSAIYGLQPNLVPEFRGVMKVDINLQKVDIDQCSSDGWFSGTHKCHLNNSECMPIKGLGFVLGAYECICKAGFYHPGVLPVNNFRRRGPDQHISGSTKDVSEEAYVCLPCREGCPFCADDSPCFVQEDKYLRLAIISFQALCMLLDFVSMLVVYHFRKAKSIRASGLILLETILFGSLLLYFPVVILYFEPSTFRCILLRWARLLGFATVYGTVTLKLHRVLKVFLSRTAQRIPYMTGGRVMRMLAVILLVVFWFLIGWTSSVCQNLEKQISLIGQGKTSDHLIFNMCLIDRWDYMTAVAEFLFLLWGVYLCYAVRTVPSAFHEPRYMAVAVHNELIISAIFHTIRFVLASRLQSDWMLMLYFAHTHLTVTVTIGLLLIPKFSHSSNNPRDDIATEAYEDELDMGRSGSYLNSSINSAWSEHSLDPEDIRDELKKLYAQLEIYKRKKMITNNPHLQKKRCSKKGLGRSIMRRITEIPETVSRQCPKEDKEGADHGTAKGTALIRKNPPESSGNTGKSKEETLKNRVFSLKKSHSTYDHVRDQTEESSSLPTESQEEETTENSTLESLSGKKLTQKLKEDSEAESTESVPLVCKSASAHNLSSEKKTGHPRTSMLQKSLSVIASAKEKTLGLAGKTQTAGVEERTKSQKPLPKDKETNRNHSNSDNTETKDPAPQNSNPAEEPRKPQKSGIMKQQRVNPTTANSDLNPGTTQMKDNFDIGEVCPWEVYDLTPGPVPSESKVQKHVSIVASEMEKNPTFSLKEKSHHKPKAAEVCQQSNQKRIDKAEVCLWESQGQSILEDEKLLISKTPVLPERAKEENGGQPRAANVCAGQSEELPPKAVASKTENENLNQIGHQEKKTSSSEENVRGSYNSSNNFQQPLTSRAEVCPWEFETPAQPNAGRSVALPASSALSANKIAGPRKEEIWDSFKV

SEQ ID NO: 3 - DNA, 3563 bp, Homo sapiens
  CDS (5)..(3553): human mGRR1b
cctcatgctggaagctctgctactctcccagtggggactccgcttctcatttcctgggccttctgttcatgatttatggatgagacagaggatgatgttggctccatctggaaagtgggtcagccagtctttgaggagacaccttggaggcctaggaatagtgctctacaccggggactcccaccagctgaagcgagccaactgctccggccgctacgagttggcgggcctgccggggaagtggccagccctggccagcgcgcacccctccttgcaccgggcgctggacacactgacacacgccaccaacttcctcaacgtgatgctgcagagcaataagtcgcgggagcagaacttgcaggacgacctggattggtaccaggcgctggtgtggagccttctggagggcgagcccagcatctcccgggcggccatcaccttcagcaccgattcgctgtccgcaccggccccacaggtcttcctccaggccacgcgcgaggagagccgcatcctgctccaagacctgtcctcctccgcaccccacctggccaacgccactctggagaccgagtggttccacggcctccggcgcaagtggaggccccacttacaccgccgcggccccaatcaggggccccggggcctgggccacagctggcggcgcaaggacgggctcggcggggacaagagccacttcaagtggtctccgccttatctggagtgcgagaacgggagttacaagcccgggtggctggttactctttcctctgccatctacgggttgcagcctaacctggtcccggaattcaggggtgtcatgaaagttgacataaatcttcagaaagtggacattgaccaatgctcaagtgatggctggttttcaggaactcataaatgccacctcaacaattcagagtgtatgccaattaaaggcctaggattcgttcttggagcctatgagtgcatttgcaaagcaggattctatcatcctggagtcttaccagtgaacaactttcggagaaggggtccggatcagcatatttcaggaagtacaaaagatgtgtcagaagaagcctatgtctgcctaccttgcagggagggctgccccttctgtgctgatgacagcccatgcttcgtccaggaagataagtatttacgacttgccatcatctccttccaagccctgtgtatgctgctcgacttcgttagcatgctggtggtctaccactttcgcaaagcaaagagcatccgggcatcgggccttatcctgttggaaacgatcctttttggatctctgctcctatactttccagttgttattttgtactttgagccaagcacatttcgctgtattctcctaagatgggctcgtcttctcggttttgctactgtttacggaactgtcactctcaaacttcacagggttttgaaggtgtttctttcacgaacggctcaacgaattccatatatgactggcggacgggtcatgaggatgctggcagtaatactcttggtagtgttttggtttctcattggctggacttcatctgtgtgccagaatttggagaaacagatttcacttattggccaggggaaaacatccgatcacctcatcttcaatatgtgcctcattgaccgctgggactacatgacagcagttgctgaatttttattcctcttgtggggtgtttatctctgctatgcagtgcggacagtcccatcggcattccatgagccccgctatatggctgttgcagttcacaatgagctcatcatctctgctatattccatacaattagatttgttcttgcctcaagacttcagtctgattggatgttgatgctgtattttgcacatactcatttgactgtgacagtcaccattgggttgcttttgattccaaagttttcacattcaagcaataacccacgagatgatattgctacagaagcatatgaggatgagctagacatgggccgatctggatcctacctgaacagcagtatcaattcagcctggagtgagcacagcttggatccagaggacattcgggacgagctgaaaaaactctatgcccaactggaaatatataaaagaaagaagatgatcacaaacaacccccacctccagaaaaagcggtgctcgaagaagggcctaggtcgttccatcatgagacgcattacggagatcccagagacagtcagccggcagtgccctaaagaggacaaggagggcgccgaccatggcacagccaaaggcactgccctcatcaggaagaaccccccagagtcttcagggaacacagggaaatccaaggaggagaccctgaaaaaccgagtcttctcactcaagaaatcccacagcacttatgaccacgtgagagaccaaacggaagagtccagtagcctacccacagaaagccaagaggaggagacaacagaaaattccacactggaatccctgtcgggtaaaaaactaacacaaaaactaaaagaagacagcgaggctgagtccacggagtcggtgccgttggtgtgcaagtcagcaagcgctcacaacctcagctcagagaagaaaactgggcacccacgaacatcgatgttacagaagtctctcagtgtcatagcaagcgccaaggagaagactcttggattagctgggaaaacccaaacagcaggtgtggaagaacgcactaaatcccagaaacctttgccaaaagataaagagacaaacagaaatcactcaaattctgataacacagagactaaagatcctgccccccaaaactcaaatcctgcggaggagccaagaaagcctcagaaatctgggattatgaaacaacaaagggtcaaccccaccactgccaattctgacctgaacccaggcaccacccagatgaaggacaactttgacattggggaggtgtgtccttgggaggtttatgacctgacccctggtcctgtgccttcagaatcaaaagttcaaaagcacgtatctattgtggcttctgaaatggagaaaaaccccactttttccttaaaggagaaatctcaccacaagcctaaggcagctgaggtttgtcagcaatccaatcagaagcgcatagataaggctgaagtatgcctttgggagagccaaggccagtccattttggaagatgagaagcttttgatttccaagactccagttctcccagagagggcaaaagaggagaacggaggtcagcctcgtgcagccaatgtgtgtgctgggcagagcgaagaactgccccccaaagctgtagcatcaaaaacagagaatgaaaatctcaaccaaataggacaccaggaaaaaaagacatcttcttctgaggagaatgtgcgtggctcctataactcaagtaataacttccagcaacctttaacatcacgagcagaggtttgtccttgggagtttgagaccccagctcaaccaaatgctggaagaagtgtagctttacctgcctcttctgctctaagtgcaaataagatagcagggcctaggaaagaagagatctgggatagttttaaagtgtagcatctcc

SEQ ID NO: 4 - protein, 1183 aa, Homo sapiens
MLEALLLSQWGLRFSFPGPSVHDLWMRQRMMLAPSGKWVSQSLRRHLGGLGIVLYTGDSHQLKRANCSGRYELAGLPGKWPALASAHPSLHRALDTLTHATNFLNVMLQSNKSREQNLQDDLDWYQALVWSLLEGEPSISRAAITFSTDSLSAPAPQVFLQATREESRILLQDLSSSAPHLANATLETEWFHGLRRKWRPHLHRRGPNQGPRGLGHSWRRKDGLGGDKSHFKWSPPYLECENGSYKPGWLVTLSSAIYGLQPNLVPEFRGVMKVDINLQKVDIDQCSSDGWFSGTHKCHLNNSECMPIKGLGFVLGAYECICKAGFYHPGVLPVNNFRRRGPDQHISGSTKDVSEEAYVCLPCREGCPFCADDSPCFVQEDKYLRLAIISFQALCMLLDFVSMLVVYHFRKAKSIRASGLILLETILFGSLLLYFPVVILYFEPSTFRCILLRWARLLGFATVYGTVTLKLHRVLKVFLSRTAQRIPYMTGGRVMRMLAVILLVVFWFLIGWTSSVCQNLEKQISLIGQGKTSDHLIFNMCLIDRWDYMTAVAEFLFLLWGVYLCYAVRTVPSAFHEPRYMAVAVHNELIISAIFHTIRFVLASRLQSDWMLMLYFAHTHLTVTVTIGLLLIPKFSHSSNNPRDDIATEAYEDELDMGRSGSYLNSSINSAWSEHSLDPEDIRDELKKLYAQLEIYKRKKMITNNPHLQKKRCSKKGLGRSIMRRITEIPETVSRQCPKEDKEGADHGTAKGTALIRKNPPESSGNTGKSKEETLKNRVFSLKKSHSTYDHVRDQTEESSSLPTESQEEETTENSTLESLSGKKLTQKLKEDSEAESTESVPLVCKSASAHNLSSEKKTGHPRTSMLQKSLSVIASAKEKTLGLAGKTQTAGVEERTKSQKPLPKDKETNRNHSNSDNTETKDPAPQNSNPAEEPRKPQKSGIMKQQRVNPTTANSDLNPGTTQMKDNFDIGEVCPWEVYDLTPGPVPSESKVQKHVSIVASEMEKNPTFSLKEKSHHKPKAAEVCQQSNQKRIDKAEVCLWESQGQSILEDEKLLISKTPVLPERAKEENGGQPRAANVCAGQSEELPPKAVASKTENENLNQIGHQEKKTSSSEENVRGSYNSSNNFQQPLTSRAEVCPWEFETPAQPNAGRSVALPASSALSANKIAGPRKEEIWDSFKV

SEQ ID NO: 5 - DNA, 8373 bp, Homo sapiens
  CDS (78)..(7178): human mGRR2
ccagtgtgctggaaagcaccgcctctgcctgggctctcctgagcctggaccccagggtcctgagagctgccccaaggatgggcaccaggggagcggtcatgccccctcctatgtgggggctgctgggctgctgttttgtctgtgcctgggctctggggggtccacggcccatccgctctctgccccctctgtcttcccaagtcaagccaggatctgtacccatgcaggtgcccctagagggggccgaggccgccctcgcttatctctactctggagatgcccagcagctatcacaggtgaattgcagtgagcgctatgaagcgcgtggggcaggagccatgccagggctccccccaagcctacagggggcagcgggcacccttgcccaggccgccaattttctcaacatgctgctgcaagccaacgacatccgtgagtccagtgtggaggaggatgtggaatggtaccaggcactggtccgcagcgtggccgagggggacccaagagtgtacagggctttgctgacctttaaccctccaccaggggccagccacctacagctggccctgcaggccacccggactggggaggaaaccatcctgcaggacttgtctgggaactgggtgcaggaggagaaccctcctggggacctggacacccctgccctgaagaagcgagtgttgaccaatgacctagggagcctcggcagccccaagtggccgcaggcagatggatatgtgggggacacgcagcaggtgaggctgtctcctcctttcctggaatgccaggagggacggctccgacctggatggctgatcacactctctgccaccttctatggactcaagccagacctcagcccagaagtcagggggcaggtgcagatggacgtagatctccagagtgtggacatcaatcagtgtgcaagtggcccaggctggtactctaacacacacctgtgtgatctcaacagcacccagtgtgttcccctggagagtcagggctttgttcttggccgctacctctgccgctgccgacctggattctacggggcaagcccctctggggggttagaggagagtgacttccagactaccgggcaattcgggttcccagaaggcagatctgggagactgctgcagtgtctgccatgtcctgagggctgcaccagctgcatggatgccacaccgtgcctggtggaagaggccgcggtgctgcgggccgctgtgctggcctgccaggcctgctgcatgctggccatcttcctgagcatgctggtctcctaccgctgccgccggaacaagaggatctgggcatctggagtggtcctgctggaaactgtcctttttggattcctgctgctttactttcctgtcttcatcctatacttcaagcccagtgtattccgctgcatcgctcttcgctgggtgcggctgctgggttttgccatcgtctacggcaccatcatactcaagctttacagagtgctgcagctgtttctgtctcgaacggcccagcggagtgcccttctgagcagcgggcggctgctgcggcgcctggggctgctcctgctacctgtgctgggcttcctggctgtgtggaccgtgggcgccctggagcgaggcatccagcacgcacctctggtgatccgaggccacactcccagtggccgccatttctacctctgtcaccacgaccgctgggactacatcatggttgtggctgagctgctgctgctgtgctggggcagcttcctctgctacgccacacgggctgtgctctcggccttccatgagccacgctacatgggcatcgccctgcacaatgagctactgctttccgctgccttccacacagccaggtttgtgctggttccctctctgcacccggactggaccctcctcctcttcttcttccacacccacagcacagtcaccaccacgctggctctgatcttcatccctaagttctggaagctgggggctcctccccgggaggagatggtggatgaggtgtgtgaggacgagctggacctgcagcactcaggctcctaccttggcagcagcatcgcctcagcctggagtgagcacagcctggaccctggagacattcgggacgagctgaagaagctctatgcccagctagaggtccacaaaaccaaggaaatggccgcaaacaacccccacctgcccaagaagcgaggcagctcatgccagggactgggccgctccttcatgaggtacctggcggaattccccgaggccctggccaggcagcactcccgggactcaggatccccaggccacggcagcctgcccggctcctcccgccgccggctcctcagctccagcctccaggaacccgaggggacaccagctctgcacaagtcccgcagcacctatgaccagcgcagggagcaggacccgcctcttcttgactcactgctgaggaggaagctggccaagaaggcctctcgaacagagagccgggagtcggtggaggggccccctgccctgggcttcaggtcagccagcgcccacaacctgacggtgggagagaggctacccagagcccggcccgcctctctgcagaagtcgctcagtgtggccagctccagggaaaaggccttgctcatggccagccaggcctacctggaggagacctaccggcaagcaaaggagcgggaggagcggaagaaggccaaggcagccatggccagcctggtgcggaggccatcagccaggaggctggagcggcctcgaggggcccccctgtcagctccaccttcccctgccaagagcagcagcgtggacagctctcacacctctgggaggcttcatgaggaggctaggagaaggctgcctcatccacccatcaggcaccaggtttctacccccatcttggccctgtctgggggcctgggagagccaaggatgctatctcccacctccaccttggctccagctctgctgccagctctagctccaaccccagcccctgccctggcaccagtcccagtatccccacaaagccccaacttactcacctacatctgcccctgggagaacgcagaactgccagccaagcaagaaaatgtgccccaggaaggcccctcagggccagagcgaggccaccactcccctgccccagctcgagccaggctctggagggccctctctgttgcagtagagaaaagcagggctggggagaatgagatggacgcagaggatgcacatcaccagagggaagctaatgatgtggacgaagacaggcccaagatcttccctaaatcccacagcctcaaggcccctgttcagcagggttccatgcgcagcctggggctggcgattaaagctctgacccgtcctcggagcacctacagagagaaggagagtgtggaggagagtcccgaggggcagaacagcgggactgcgggagagagtatgggggcaccctcccgatcgcccaggctaggccggcccaaggcggtgagtaagcaggccgctcttatcccctccgatgacaaggagtccctccagaaccaacagaacgctcacaccagcaggatgctccaagtctgtcaacgggagggcagcagggaacaagaagacagaggcaggaggatgacccagggtctaggggaacggaaagctgagagagcaggtaaaacagggcttgccatgctgaggcaagtttccagggacaaaaacatcaagcaatcaaaagaaacccctgtcgggtggcaggaactgcccaaagctggcctccagtccctcggcagcgctgaccacagggtggcagaggtatgcccctgggaggtcactgaatcagaaacgcgtcagccagacagtggcaacaaggccgaaatctgcccctgggagacgagtgaaggagccccagagtcgagggcactaagacaagacccaggtgactcccaaaaaaagagaggggaggcccggggaaaatcagagcccatagatgtggttcccatgatgcggaaaaagccagagaggctggtgagggagcaggaagcagtgtgtccctgggagagtgccgatcgaggaggtctgtcccctgggtcagctcctcaggaccctggcagaatcagagacaaatctgaggcgggggacagtgtggaggccaggaaggtggagaagcctgggtgggaagctgctggcccagaagctcatacccctgacatcaccaaggcagagccgtgtccctgggaggcaagtgaaggaggcgaggatgggaaaccagcccaagaggcagtgaaggatctccctcaggaaaagcagaaaaccaggaaagcaaccttttggaaagaacagaaaccgggaggagacttggagtctctttgtccatgggagagtacagatttccggggcccctcagcagtctcaattcaggccccaggaagctcagagtgttcagggagtttgggcagtggcattgctgaagtgtgtctgtgggaggcaggagatgctcctgctatccagaaagcagagatctgtccctgggagctggatgataacgtgatggggcaggaaatgctgagtctggggacaggtagagaatctcttcaagaaaaggaaaaagcctccagaaaaggaagctttggagagatgggggaacaaactgtgaaagcagtgcagaaattaagtcaacagcaggagtcagtttgtcccagggagagcacggtccctgggcactccagcccatgtctagacaattcctcatccaaagctggtagccaattcctatgcaatggaggaagcagagcaacgcaggtgtgtccacaggaagatctcaggccggaggcacaggaagcaacacctgccaaaacagaaatctgtccctgggaggtaaatgaaagaacaagagaggaatggacatcagcacaggtgccaagaggaggagaatctcaaaaggacaaggagaaaatgcctggaaaatcggaaatcgaagatgtcacagcttgggaaaagcctgaggggcagatccaaaagcaagaagcggtcggcccctgggagagtgtggaccctggcagcttctccccacaaccacgtcctcaagacacagagagaccccaaacccttctccagatgtcaggcagtgtgggaagcaaagctgccgacatttgccctttggatgtggaggaaaacttgactgctgggaaggcagaaatctgtccctgggaggtgggtgctggagcaggggaggaaagggctttgggagctgaggccattaggaaatctccaaatgatacaggcaaggtttctgcagatcttggacccagggagagagctgttactgctccagagaagccacagaagccaaccccagagtgggaggtggcttgtccctgggggagtgtgggtccaggggcctgttctcagcatccaggtactctagatgctgatggaccaaaagctgggttccaggaactggatcatatgggctgcaggccaggtgaagtgtgtccctgggaagcacaggaagctgctaccagtgaaaaagccaagatctgtccctgggaggtaagtgaaggaactactgggaagggattggaccaaaaggcagggagtgaatcagcagagcagagggagaaagctctagaaaaggggagactcacttccctgggagaagacgtatcaaaagggatggcaaaactgtgtcaacaacaggaaactatttgtatttgggagaacaaggacttgagggaatcccctgctcaggcccccaagatctcagacttgcccagcagcatgagtagtgaagtggcagagggacattccttggaagcaacagagaagggggacctgagacaagacccaaagacaggttccttcccagaacacataacccaagaaaaagctccagctgcagacacagaagaattcactactgaagatggggaaaaaacaagccatgagctacaatccgtctgtccatgggagaccactgccccagcagattccgtctctcacctagacagacagcgccctgaccaacctaaagctagctcccagagactggtcagcactgggggcagggccgctgacgtgtgcccatgggatgttcctgatgcaggtgtgtataaatctgacagcagtgccaaggctgagacctgtccctgggaagtgactgaaagaatccctgtcaaaggggtgtcaaggcaggatggaaaaggggactctcaagaagagaaaggcagagccccagaaaaatcagagccaaaaggtgtgccagttcagaaaaagccagagatggcagacttcaggcagcaggaggctgtgtgtccctgggagagtcaagatggcaagggtctgtccccacagccagccccagatgcttctgacagaagcagaggcagttctgaggcagcaggcagtgtggagaccagggtagcggaagtgtgtctgtgggaagtggtagaggctccctctgccaagaaagcagagatctgcccttgggaggcgggtggaggagcagcagaggaaggggaacaggaaagagaatcacaagggcaaggagagatgttccttcagaaggcaggacctggagggacggaagaacacttctcaaaagcagcagcaaagcccagagagcaggaggcagtctgccctggggaaggcacaggctcaggagggctcttgccccagtcaggtgccctggacccagaactcaaagtcagccccaaggaagcaggcagcatgggaagcaggatggcagagctgtgccaatgggaaatcacagatccagaaggaaataaaataaagggtaccatggcagacatctgtcctggggaggaaactggagtcccatctgaggaatctggcctcctggctttaacagcaactcggagagaatttttccccacagctcctgaaaaaccactatgccttttagtccatgggcctctggatcacttctttccagaaagcaaaatcccctgccccaaggtaagcaggccagccagtactttcactctagaaggtgtcagagaactacaaggaccttcagggcttgagccaaggaccagcttagccccagagccaagtctccaggaagctgagtctcagtcttcgtccttaactgaagactcaggccaagtggcttttgaagctcagtatgaagaattcacccctcccactgtctatccttgggattgggagtaacagccttattaggtgaggtcaaacacagagctagctttcaggagccaaggccctttccaagtccaggtgttcccaaagagctgaatcaaagacagcttggccacttcccctccttgagaagaccagaagtcaacccatcccaaagacaaactgcatgaaaagggtacagcttagaccccaatgggaaggcccaccctctctttacttctaacttttcttcctgtttgaggaaacaaagactggacactctacttctaaggacgcctcccatccacctacagtactcacaacacaaaccacccgagtctggacactcacccacaaagccactgttagggatgaaaaaagtcagttgtgcccagcctaccttctctcacagggggacccaattctctgaagtctacgatggagcattttttgagaagaaactaaaccatacaaagtgttggcaacgtagggatggtggaccacagaaggctataaaggccaccatagaaagctgaagagccatttaattgaagcggaagataaattgggggaaaagctaaaatgactcaaaataggggcctgaaaatgctttaaggaccttgtcattttgactttgaatctcctgtttctgctttattgcttctcatacctgccctgcttgactgctgaagcagaaacaatcaggcacgctgacacccttatcagcatctggcatccaaagcaacatcagagagagagaacacagaagaaaagcaattgccccaggtcctccccacagtcaagggagctcttccttccagacatccttgtgtgcctcttgctagttttttagggggtctccccctcttcctgccctagctctcaccagtctccagagctcgtctcactggctcatttccttccccctagcttttcagcgctgccatcaacaggacctctctcttttgtcaaggcaactggagtcaaggggaatctctggttctgatgcaagacccatccaccctcccccacccctccccgcccaagaatcacttgctcagtaacaagaggccacatcaatctccttccagaggagctcaagaaaggcagtcctgtacacagccagcactctacttcttctgccctctaaaagaggagtgctacagggcaagttagctttccagcacagtggcggccgctcgagtctagagggcccgtttaaacc

SEQ ID NO: 6 - protein, 2367 aa, Homo sapiens
MGTRGAVMPPPMWGLLGCCFVCAWALGGPRPIRSLPPLSSQVKPGSVPMQVPLEGAEAALAYLYSGDAQQLSQVNCSERYEARGAGAMPGLPPSLQGAAGTLAQAANFLNMLLQANDIRESSVEEDVEWYQALVRSVAEGDPRVYRALLTFNPPPGASHLQLALQATRTGEETILQDLSGNWVQEENPPGDLDTPALKKRVLTNDLGSLGSPKWPQADGYVGDTQQVRLSPPFLECQEGRLRPGWLITLSATFYGLKPDLSPEVRGQVQMDVDLQSVDINQCASGPGWYSNTHLCDLNSTQCVPLESQGFVLGRYLCRCRPGFYGASPSGGLEESDFQTTGQFGFPEGRSGRLLQCLPCPEGCTSCMDATPCLVEEAAVLRAAVLACQACCMLAIFLSMLVSYRCRRNKRIWASGVVLLETVLFGFLLLYFPVFILYFKPSVFRCIALRWVRLLGFAIVYGTIILKLYRVLQLFLSRTAQRSALLSSGRLLRRLGLLLLPVLGFLAVWTVGALERGIQHAPLVIRGHTPSGRHFYLCHHDRWDYIMVVAELLLLCWGSFLCYATRAVLSAFHEPRYMGIALHNELLLSAAFHTARFVLVPSLHPDWTLLLFFFHTHSTVTTTLALIFIPKFWKLGAPPREEMVDEVCEDELDLQHSGSYLGSSIASAWSEHSLDPGDIRDELKKLYAQLEVHKTKEMAANNPHLPKKRGSSCQGLGRSFMRYLAEFPEALARQHSRDSGSPGHGSLPGSSRRRLLSSSLQEPEGTPALHKSRSTYDQRREQDPPLLDSLLRRKLAKKASRTESRESVEGPPALGFRSASAHNLTVGERLPRARPASLQKSLSVASSREKALLMASQAYLEETYRQAKEREERKKAKAAMASLVRRPSARRLERPRGAPLSAPPSPAKSSSVDSSHTSGRLHEEARRRLPHPPIRHQVSTPILALSGGLGEPRMLSPTSTLAPALLPALAPTPAPALAPVPVSPQSPNLLTYICPWENAELPAKQENVPQEGPSGPERGHHSPAPARARLWRALSVAVEKSRAGENEMDAEDAHHQREANDVDEDRPKIFPKSHSLKAPVQQGSMRSLGLAIKALTRPRSTYREKESVEESPEGQNSGTAGESMGAPSRSPRLGRPKAVSKQAALIPSDDKESLQNQQNAHTSRMLQVCQREGSREQEDRGRRMTQGLGERKAERAGKTGLAMLRQVSRDKNIKQSKETPVGWQELPKAGLQSLGSADHRVAEVCPWEVTESETRQPDSGNKAEICPWETSEGAPESRALRQDPGDSQKKRGEARGKSEPIDVVPMMRKKPERLVREQEAVCPWESADRGGLSPGSAPQDPGRIRDKSEAGDSVEARKVEKPGWEAAGPEAHTPDITKAEPCPWEASEGGEDGKPAQEAVKDLPQEKQKTRKATFWKEQKPGGDLESLCPWESTDFRGPSAVSIQAPGSSECSGSLGSGIAEVCLWEAGDAPAIQKAEICPWELDDNVMGQEMLSLGTGRESLQEKEKASRKGSFGEMGEQTVKAVQKLSQQQESVCPRESTVPGHSSPCLDNSSSKAGSQFLCNGGSRATQVCPQEDLRPEAQEATPAKTEICPWEVNERTREEWTSAQVPRGGESQKDKEKMPGKSEIEDVTAWEKPEGQIQKQEAVGPWESVDPGSFSPQPRPQDTERPQTLLQMSGSVGSKAADICPLDVEENLTAGKAEICPWEVGAGAGEERALGAEAIRKSPNDTGKVSADLGPRERAVTAPEKPQKPTPEWEVACPWGSVGPGACSQHPGTLDADGPKAGFQELDHMGCRPGEVCPWEAQEAATSEKAKICPWEVSEGTTGKGLDQKAGSESAEQREKALEKGRLTSLGEDVSKGMAKLCQQQETICIWENKDLRESPAQAPKISDLPSSMSSEVAEGHSLEATEKGDLRQDPKTGSFPEHITQEKAPAADTEEFTTEDGEKTSHELQSVCPWETTAPADSVSHLDRQRPDQPKASSQRLVSTGGRAADVCPWDVPDAGVYKSDSSAKAETCPWEVTERIPVKGVSRQDGKGDSQEEKGRAPEKSEPKGVPVQKKPEMADFRQQEAVCPWESQDGKGLSPQPAPDASDRSRGSSEAAGSVETRVAEVCLWEVVEAPSAKKAEICPWEAGGGAAEEGEQERESQGQGEMFLQKAGPGGTEEHFSKAAAKPREQEAVCPGEGTGSGGLLPQSGALDPELKVSPKEAGSMGSRMAELCQWEITDPEGNKIKGTMADICPGEETGVPSEESGLLALTATRREFFPTAPEKPLCLLVHGPLDHFFPESKIPCPKVSRPASTFTLEGVRELQGPSGLEPRTSLAPEPSLQEAESQSSSLTEDSGQVAFEAQYEEFTPPTVYPWDWE

SEQ ID NO: 7 - DNA, 27 bp, Artificial
  primer_bind (1)..(27): gene-specific primer (5′-RACE)
ccgcactgcatagcagagataaacacc

SEQ ID NO: 8 - DNA, 29 bp, Artificial
  primer_bind (1)..(29): gene-specific primer (3′-RACE)
caatgagctcatcatctctgctatattcc

SEQ ID NO: 9 - DNA, 27 bp, Artificial
  primer_bind (1)..(27): RACE primer designed from sequence GA_15234422
gcggggctcatggaatgccgatgggac

SEQ ID NO: 10 - DNA, 27 bp, Artificial
  primer_bind (1)..(27): RACE primer designed from sequence GA_15234422
gtcccatcggcattccatgagccccgc

SEQ ID NO: 11 - DNA, 28 bp, Artificial
  primer_bind (1)..(28): PCR primer used for mGRR1a
caccatggcttaccccttactcctctgc

SEQ ID NO: 12 - DNA, 28 bp, Artificial
  primer_bind (1)..(28): PCR primer used for mGRR1a
cgttgttgctcttgcccccctggtcctc

SEQ ID NO: 13 - DNA, 27 bp, Artificial
  primer_bind (1)..(27): PCR primer used for mGRR1b
gtgggaccagctgtgctgccattgatc

SEQ ID NO: 14 - DNA, 28 bp, Artificial
  primer_bind (1)..(28): PCR primer used for mGRR1b
cgttgttgctcttgcccccctggtcctc

SEQ ID NO: 15 - protein, 17 aa, Homo sapiens
  SIGNAL (1)..(17)
MAYPLLLCLLLAQLGLG

SEQ ID NO: 16 - DNA, 816 bp, Rattus norvegicus
  Unsure (1)..(816): Partial sequence of rat mGRR1a
aatggaagtcagttgtacacaaccaatatgtgtgctgggcaatatgaagaactgccccccaaagccgtagcatcaaaagtagagaatgaaaatctcaaccaaatgggagaccaggagaaacagacatcttcttctgtggatatcatccctggctcctgtatctcgagtaataactcccctcaacccttaacgtcacgagccgaagtgtgtccctgggagttcgagcccctagaacaaccaaatgctgaaagaatcgtagctttacccgcctcctctgctttaagtgcaagtaagatacccgggccacggaaataggaagtccaggacactttcaaaatgtaccaccctgggaagaaaaggggtaatcccgagtttgataaaaatagatctgaaaatcaaacgttaccaaggaatctttgttagcctagggaaacatggcacatggtgcggtaaggtgaaggcaagggctggagaaggccagaaaaaggcagtaatgtgtgcctccagccatgaaaaaccagatcaacactgggggacagcttagcatttcagcctgttaaagggacatttccctgtctgtctttacaagtgggattgaatggccccaagcaagattcaccaggaggcatggaagacaggggcatggattttatgatggataaacttagatgcacgtgactgtaattctaagtagtcgactgaaaagaccttcatcacctatttaaccttggtagcactgctaacttaatgcaccccagtaccttttatatacacgattgctctcattttcttaaaatctgtgtgtgtcagtaaatt

SEQ ID NO: 17 - DNA, 20 bp, Artificial
  primer_bind (1)..(20): PCR primer with attached T7 I promotor sequences
aatggaagtcagttgtacac

SEQ ID NO: 18 - DNA, 40 bp, Artificial
  primer_bind (1)..(40): PCR primer with attached T7 I promotor sequences
taatacactcactatagggaaatgtccctttaacaggctg

SEQ ID NO: 19 - DNA, 41 bp, Artificial
  primer_bind (1)..(41): PCR primer with attached T7 I promotor sequences
taatacgactcactatagggaaatggaagtcagttgtacac

SEQ ID NO: 20 - DNA, 20 bp, Artificial
  primer_bind (1)..(20): PCR primer with attached T7 I promotor sequences
aatgtccctttaacaggctg

SEQ ID NO: 21 - DNA, 27 bp, Artificial
  sig_peptide (1)..(27): C-terminal hemagglutinin (HA) epitope
tatccatatgatgttccagattatgct

SEQ ID NO: 22 - DNA, 22 bp, Artificial
  primer_bind (1)..(22): PCR primer made by using human mGRR1a in pcDNA3.1 topo as template
caagactccagttctcccagag

SEQ ID NO: 23 - DNA, 64 bp, Artificial
  primer_bind (1)..(64): PCR primer made by using human mGRR1a in pcDNA3.1 topo as template
tctagatctagactaagcataatctggaacatcatatggatacactttaaaactatcccagatc

SEQ ID NO: 24 - DNA, 28 bp, Artificial
  primer_bind (1)..(28): RACE primer designed from sequence GA_15091955
cccgctgctcagaagggcactccgctgg

SEQ ID NO: 25 - DNA, 27 bp, Artificial
  primer_bind (1)..(27): RACE primer designed from sequence GA_15091955
tggaccgtgggcgccctggagcgaggc

SEQ ID NO: 26 - DNA, 27 bp, Artificial
  primer_bind (1)..(27): PCR primer
gctgggactacatcatggttgtggctg

SEQ ID NO: 27 - DNA, 28 bp, Artificial
  primer_bind (1)..(27): PCR primer
gggccgttcgagacagaaacagctgcag

SEQ ID NO: 28 - DNA, 25 bp, Artificial
  primer_bind (1)..(25): PCR primer
caccgcctctgcctgggctctcctg

SEQ ID NO: 29 - DNA, 26 bp, Artificial
  primer_bind (1)..(26): PCR primer
cgctgctcagaagggcactccgctgg

SEQ ID NO: 30 - DNA, 27 bp, Artificial
  primer_bind (1)..(27): PCR primer
ggattcctgctgctttactttcctgtc

SEQ ID NO: 31 - DNA, 24 bp, Artificial
  primer_bind (1)..(24): PCR primer
ccacagacacacttcagcaatgcc

SEQ ID NO: 32 - DNA, 27 bp, Artificial
  primer_bind (1)..(27): PCR primer
ccaggaaggtggagaagcctgggtggg

SEQ ID NO: 33 - DNA, 24 bp, Artificial
  primer_bind (1)..(24): PCR primer
ctaacttgccctgtagcactcctc

SEQ ID NO: 34 - protein, 27 aa, Homo sapiens
  SIGNAL (1)..(27)
MGTRGAVMPPPMWGLLGCCFVCAWALG